(19) United States
(12) Patent Application Publication
(10) Pub. No.: US 2024/0385202 A1
(43) Pub. Date: Nov. 21, 2024

(54) SYSTEMS, METHODS, AND APPARATUSES FOR IMPROVED INSTRUMENT MONITORING AND ANALYTICS

(71) Applicant: Regeneron Pharmaceuticals, Inc., Tarrytown, NY (US)

(72) Inventors: Vinay Desai, Tarrytown, NY (US); John McLoughlin, Tarrytown, NY (US); Cristian Pelle, Tarrytown, NY (US); Michael Georgiadis, Tarrytown, NY (US)

(21) Appl. No.: 18/634,633

(22) Filed: Apr. 12, 2024

Related U.S. Application Data
(60) Provisional application No. 63/502,750, filed on May 17, 2023.

(51) Int. Cl. G01N 35/00

(52) U.S. Cl. CPC G01N 35/00712; G01N 35/00871

(57) ABSTRACT

An instrument device may host or execute one or more instrument applications configured for transferring input data collected during studies, process-oriented workflows and/or experiments to a monitoring and analytics system via a support device. However, the format of such input data may be proprietary for that particular instrument device and therefore less beneficial in an environment where various types of instrument devices are used. To resolve the issues inherent in managing a multitude of instrument devices and proprietary data formats, utilization data of support devices that operate and control the instrument devices may be used as a proxy to determine current or historical utilization rates/activity levels for the instrument devices.

### CROSS-REFERENCE TO RELATED APPLICATION(S)

This application claims the priority benefit, under 35 U.S.C. 119 (e), of U.S. Provisional Patent Application No. 63/502,750, filed May 17, 2023, which is incorporated herein by reference in its entirety for all purposes.

### BACKGROUND

Biomedical research at large scales can span hundreds of labs, each potentially having multiple instruments used multiple times daily by thousands of researchers. Experiments performed using these instruments may span hours, days, weeks, or months, and may depend heavily on availability and continuity between instruments. Instrument usage and uptime plays a critical role in successful experimental outcomes; however, these instruments may be manufactured by dozens or hundreds of different manufacturers each offering proprietary and specific monitoring capabilities, making singular monitoring and coordination across all instruments difficult and cumbersome. No singular, efficient solution exists for providing instrument users and administrators with an accurate indication of instrument status and/or for monitoring a wide variety of instruments for maintenance and usage patterns.

### SUMMARY

An instrument device may host or execute one or more instrument applications configured for transferring input data collected during studies, process-oriented workflows and/or experiments to a monitoring and analytics system. However, the format of such input data may be proprietary for that particular instrument device and therefore less beneficial in an environment (e.g., laboratory) where various types of instrument devices are used. To resolve the issues inherent in managing a multitude of instrument devices and proprietary data formats, utilization data associated with support devices that operate and control the instrument devices may be used as a proxy to determine current or historical use of the instrument devices.

In one example, the inventive concepts disclosed herein are implemented in a research laboratory environment including a plurality of instrument devices respectively operated by a plurality of researchers to conduct studies, process-oriented workflows and/or experiments. In such a research laboratory environment, one example of the inventive concepts is directed to a method for monitoring an operating status of respective instrument devices of the plurality of instrument devices and communicating to the plurality of researchers the operating status of the respective instrument devices to facilitate efficient utilization of the plurality of instrument devices. In some aspects, the method further may significantly mitigate interruptions in the studies, process-oriented workflows and/or experiments, and improve continuity and reliability of data collected by the respective instrument devices during the studies, process-oriented workflows and/or experiments is disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

In some aspects, the techniques described herein relate to a method for managing a plurality of instrument devices communicatively coupled to respective support devices of a plurality of support devices, the method including: receiving, from a plurality of support devices and a plurality of instrument devices, utilization data for each support device of the plurality of support devices and each instrument device of the plurality of instrument devices, the plurality of support devices being communicably coupled to the plurality of instrument devices, wherein the utilization data is associated with execution of at least one instrument application on each instrument device of the plurality of instrument devices, and wherein each support device controls the instrument device to which that support device is communicably coupled via the at least one instrument application executing on that instrument device; generating structured utilization data for each support device of the plurality of support devices and each instrument device of the plurality of instrument devices, wherein the structured utilization data includes a plurality of fields; determining, based on at least one query associated with the structured utilization data, a status for each instrument device of the plurality of instrument devices, wherein the at least one query is associated with at least one field of the plurality of fields; and outputting, via an information device, an indication of the status for each instrument device of the plurality of instrument devices.

In some aspects, the techniques described herein relate to a method for managing an instrument device, the method including: performing, by an instrument device, a physical operation corresponding to an experiment; monitoring, by a support device communicatively coupled to the instrument device, sensor data corresponding to an operational parameter representing the physical operation for the instrument device; calculating, by the support device, a target value for the operational parameter based on the sensor data; calculating, by the support device, a standard deviation threshold based on the sensor data; identifying, by the support device, one or more data points of real-time sensor data exceeding the standard deviation threshold; and performing, by the support device, an action in response to the one or more data points of real-time sensor data exceeding the standard deviation threshold.

In some aspects, the techniques described herein relate to a method, including: receiving, from a plurality of support devices, utilization data for each support device of the plurality of support devices, the plurality of support devices being communicably coupled to a plurality of instrument devices, wherein the utilization data is associated with execution of at least one instrument application on each instrument device of the plurality of instrument devices, and wherein each support device controls the instrument device to which that support device is communicably coupled via the at least one instrument application executing on that instrument device; generating, based on the utilization data for each support device of the plurality of support devices, and based on a database schema associated with the plurality of instrument devices, a modified database schema including a plurality of fields to facilitate querying the utilization data associated with a specific instrument device of the plurality of instrument devices; generating, based on the modified database schema and the utilization data for each support device of the plurality of support devices, structured utilization data for each support device of the plurality of support devices, wherein the structured utilization data includes the plurality of fields; determining, based on at least one query associated with the structured utilization data for each support device of the plurality of support devices, a utilization rate for each instrument device of the plurality of instrument devices, wherein the at least one query is associated with at least one field of the plurality of fields; and outputting, via a graphical user interface, an indication of the utilization rate for each instrument device of the plurality of instrument devices.

In some aspects, the techniques described herein relate to a method including: receiving, via a plurality of support devices, a plurality of log files associated with a plurality of instrument devices, the plurality of instrument devices each executing at least one instrument application, wherein each support device of the plurality of support devices is communicably coupled to at least one instrument device of the plurality of instrument devices, and wherein each support device controls the at least one instrument device to which that support device is communicably coupled via the at least one instrument application executing on that instrument device; determining, based on the plurality of log files, a plurality of instrument-specific utilization metrics for each instrument device of the plurality of instrument devices; determining, based on the plurality of instrument-specific utilization metrics for each instrument device of the plurality of instrument devices, a health indicator for each instrument device of the plurality of instrument devices, wherein the health indicator is indicative of an availability of the corresponding instrument device; and outputting, via an information device, the health indicator for each instrument device of the plurality of instrument devices.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows an example system for improved instrument monitoring and analytics.

FIG. 2 shows example raw utilization data for an example support device.

FIG. 3. illustrates raw utilization data ingested by the monitoring and analytics system in just one day.

FIG. 4 shows an example of structured utilization data for four example support devices based on corresponding raw utilization data.

FIG. 5 shows an example of a Processor-based Utilization Query with annotations to describe various aspects/sub-queries of the Processor-based Utilization Query.

FIG. 6 shows an example Memory-based Utilization Query.

FIG. 7 shows an example of an instrument-specific log.

FIG. 8 illustrates an example of an instrument management software information.

FIG. 9 shows an example of an analytics schema.

FIG. 10 shows an example dashboard as well as example visualizations that may be generated by a dashboard module.

FIG. 11 shows an example of the instrument-specific utilization data, which may be output as one or more verbose logs.

FIG. 12 shows example results of a query of instrument-specific utilization data with instrument-specific filters.

FIG. 13 shows an example health dashboard that the dashboard module may output based on results of queries using the fields extracted from the verbose logs.

FIG. 14 shows another example health dashboard with a map/diagram of example rooms that may house one or more example instrument devices and support devices.

FIG. 15 shows a block diagram depicting a system including a computing device and a server connected through a network.

FIG. 16 shows a flowchart of an example method for improved instrument monitoring and analytics.

FIG. 17 shows a flowchart of an example method for improved instrument monitoring and analytics.

FIG. 18A illustrates an example of a first information device configured to present information such as exemplary dashboards described herein in an office environment.

FIG. 18B illustrates another example of a second information device configured to present information such as exemplary dashboards described herein in a common area (e.g., a hallway) connecting multiple research laboratory and/or office environments.

FIG. 18C illustrates another example of a third information device configured to present information such as exemplary dashboards described herein in a laboratory environment.

FIG. 19 illustrates a dashboard for monitoring one or more support devices and providing device telemetry in accordance with the present technology.

FIG. 20 shows a dashboard for monitoring sensor inputs for an exemplary instrument device over time.

FIG. 21 illustrates time series temperature data ingested by monitoring and analytics system from temperature sensor disposed in a biological sample freezer and presented as a dashboard on a display.

FIG. 22 shows a flowchart of an example method for improved instrument monitoring and analytics.

FIG. 23 shows a flowchart of an example method for improved instrument monitoring and analytics.

### DETAILED DESCRIPTION

The disclosed methods and systems may be understood more readily by reference to the following detailed description of particular embodiments and the Example included therein and to the Figures and their previous and following description.

It is understood that the disclosed method and systems are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a.”, “an.” And “the” include plural reference unless the context clearly dictates otherwise. Thus, for example, reference to “an image” includes a plurality of images, and so forth.

“Optional” or “optionally” means that the subsequently described event, circumstance, or material may or may not occur or be present, and that the description includes instances where the event, circumstance, or material occurs or is present and instances where it does not occur or is not present.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Exemplary” means “an example of” and is not intended to convey an indication of a preferred or ideal configuration. “Such as” is not used in a restrictive sense, but for explanatory purposes.

Ranges may be expressed herein as from “about” one particular value, and/or to “about” another particular value. When such a range is expressed, also specifically contemplated and considered disclosed is the range-from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Described herein are methods, systems, and apparatuses for improved instrument monitoring and analytics. An instrument device may host or execute one or more instrument applications configured for transferring “input data” collected or generated during studies, process-oriented workflows and/or experiments, which may be stored at a monitoring and analytics system (e.g., via a support device). Each support device may comprise one or more support device applications configured for controlling/operating the instrument device(s) in communication with that particular support device via the one or more instrument applications. A support device application may generate and/or collect various types of “performance data” during operation. The performance data may be associated with the support device's control/operation of the corresponding instrument device(s) via the one or more support device applications and the one or more instrument applications. The performance data may also be associated with the support device receiving, processing, and/or sending the input data (e.g., for storage of the input data). Such performance data is referred to herein as “raw utilization data” or simply “utilization data,” which may be indicative of an amount of resources the corresponding support device is using at a specific time and/or over a configurable period of time. The instrument devices may be capable of transferring input data that relates to an experiment or study to a corresponding support device; however, the instrument devices may not be capable of (or configured for) sending the input data in a manner that would indicate their current or historical utilization rate/activity. For example, for some instrument devices, the corresponding one or more instrument applications may be capable of collecting and/or generating a form of utilization data, but the format of such data may be proprietary for that particular instrument device and therefore less beneficial in an environment (e.g., laboratory) where various types of instrument devices are used.

To resolve the issues inherent in managing a multitude of instrument devices and proprietary data formats, the raw utilization data associated with the support devices may be used as a proxy to determine current or historical utilization rates/activity levels for the various instrument devices. For example, when an instrument device sends input data to a corresponding support device (e.g., experiment-related data), as well as when the support device controls/operates the instrument device, the corresponding raw utilization data may indicate a level of processor(s) use, memory use, etc., for the support device that serves as a proxy or heuristic for utilization of the instrument device.

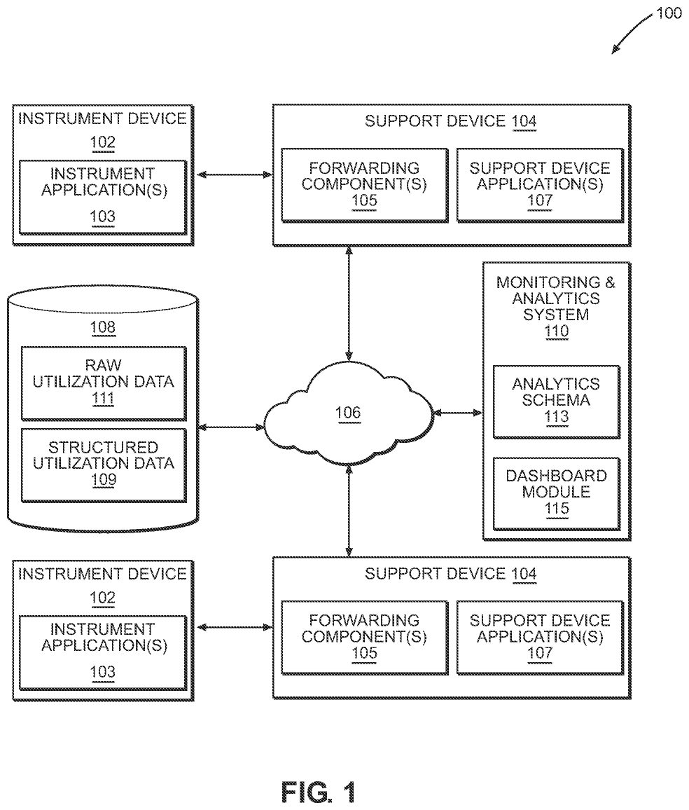

FIG. 1 shows an example system 100 for improved instrument monitoring and analytics. Those skilled in the art would understand that FIG. 1 represents one example of a system and is not meant to be limiting. The system 100 may comprise one or more support devices 104 that are in communication with one or more instrument devices 102. An instrument device 102 may comprise, but is not limited to, any one of a variety of laboratory instruments or equipment, such as a microscope, flow cytometer, spectrometer, scanner, protein analyzer, etc. An instrument device may perform any suitable operation/set of operations including, but not limited to, one or more biological operations (e.g., fermentation, cell culture, etc.), chemical operations (e.g., purification, crystallization, etc.), physical operations (e.g., thermal, acoustic, mechanical, electrical, magnetic, electromagnetic, optical, fluidic, etc.) such as increasing or decreasing a temperature of an object, moving (e.g., spinning, lifting, shaking, rotating, tilting, stretching, compressing, or any suitable movement) an object, applying a voltage to an object, applying a current to an object, emitting electromagnetic radiation at an object, defining or setting a frequency of an object, or any suitable physical operation. The one or more operations may correspond to an experiment, such as a scientific experiment, an in vivo animal experiment, a biological experiment, an in vitro experiment, or any suitable experiment.

A support device 104 may comprise a computing device, such as a laptop, desktop, tablet, mobile device, etc. A support device 104 and an instrument device 102 may be in communication by wired or wireless means. A support device 104, which is capable of network communications, may function as a bridge between a corresponding instrument device 102 and a monitoring and analytics system 110. Support devices 104 may be in communication with the monitoring and analytics system 110 via a network 106. The network 106 may comprise, for example, one or more LANs, WANs, cellular networks (e.g., LTE, HSPA, 3G, and other cellular technologies), and/or networks using any of wired, wireless, terrestrial, microwave, or satellite links, and may include the Internet.

An instrument device 102 may host or execute one or more instrument applications 103 configured for transferring “input data” collected or generated during studies, process-oriented workflows and/or experiments to the monitoring and analytics system 110 via a support device 104. Each support device 104 may comprise one or more support device applications 107 configured for controlling/operating the instrument device(s) 102 in communication with that particular support device 102 via the one or more instrument applications 103. A support device application 107 may generate and/or collect various types of “performance data” during operation of the corresponding support device 104, such as events, logs, network data, sensor data, and/or other types of machine-generated data. The performance data may be associated with the support device's 104 control/operation of the corresponding instrument device(s) 102 via the one or more support device applications 107 and the one or more instrument applications 103. The performance data may also be associated with the support device 104 receiving, processing, and/or sending the input data (e.g., for storage of the input data at the monitoring and analytics system 110). Such performance data is referred to herein as “raw utilization data,” or simply “utilization data” (e.g., machine-generated raw data, Windows Management Instrumentation™ events or logs, etc.). For example, the raw utilization data 111 may be indicative of an amount of resources the corresponding support device 104 is using at a specific time and/or over a configurable period of time, such as processor(s) use, non-volatile memory use (e.g., read-only memory), volatile memory use (e.g., random access memory (RAM)), storage device(s) use (e.g., capacity, size, etc.), a combination thereof, and/or the like.

The instrument devices 102 may be capable of transferring input data that relates to an experiment or study to a corresponding support device 104; however, the instrument devices 102 may not be capable of (or configured for) sending the input data in a manner that would indicate their current or historical utilization rate/activity. For example, for some instrument devices 102, the corresponding instrument application 103 may be capable of collecting and/or generating a form of utilization data, but the format of such data may be proprietary for that particular instrument device 102 and therefore less beneficial in an environment (e.g., laboratory) where various types of instrument devices 102 are used. To resolve the issues inherent in managing a multitude of instrument devices 102 and proprietary data formats, the raw utilization data 111 associated with the support devices 104 may be used as a proxy to determine current or historical utilization rates/activity levels for the various instrument devices 102. For example, when an instrument device 102 sends input data to a corresponding support device 104 (e.g., experiment-related data via an instrument application 103), the corresponding raw utilization data 111 may indicate a level of processor(s) use, memory use, etc., for the support device 104 that serves as a proxy or heuristic for utilization of the corresponding instrument device 102.

Raw utilization data 111 associated with one or more of the instrument devices 102 may be sent by the support devices 104, via the network 106, to a database 108. The database 108 may comprise configuration files/information for the instrument devices 102 and/or the support devices 104. The database 108 may receive raw utilization data 111 associated with an instrument device 102 and generate (e.g., via a server(s)-not shown) structured utilization data 109. The structured utilization data 109 for a particular support device 104 may be based on the raw utilization data 111 corresponding to that support device 104. The structured utilization data 109 may comprise aspects or portions of the raw utilization data 111, except the structured utilization data 109 may have a higher degree of organization according to a database schema. The structured utilization data 109 may be appended to a relational database(s) within the database 108 according to the database schema to enable querying of the structured utilization data 109 via the monitoring and analytics system 110, which is further discussed herein.

The support devices 104 may each comprise a forwarding component 105. The forwarding component 105 may comprise software or other logic that facilitates sending the raw utilization data 111 for a corresponding support device 104 to the database 108 and/or to the monitoring and analytics system 110. For example, as described herein, the support device application 107 may generate and/or collect the raw utilization data 111″ (e.g., machine-generated raw data) for the corresponding support device 104 (e.g., processor(s) use, memory use, etc.), and the forwarding component 105 may be responsible for sending that raw utilization data 111 to the database 108 and/or to the monitoring and analytics system 110 via the network 106. In some examples, the forwarding component 105 for a particular support device 104 may collect instrument-specific information corresponding to the instrument device 102. Such instrument-specific information may comprise, for example, an identifier(s) for the instrument device 102, a type of the instrument device 102, a manufacturer and/or model of the instrument device 102, a version(s) of the instrument application 103, information related to an operating system for the instrument device 102, etc. In some examples, the forwarding component 105 may collect support device-specific information corresponding to the particular support device 104. Such support device-specific information may comprise, for example, an identifier(s) for the support device 104, a type of the support device 104, a manufacturer and/or model of the support device 104, a version(s) of the support device application 107, information related to an operating system for the support device 104, etc.

As shown in FIG. 1, the forwarding component 105 is separate from the support device application 107. However, in some configurations of the system 100, the forwarding component 105 may be a component of the support device application 107, a plug-in, an extension, or any other type of add-on component. The forwarding component 105 may comprise, in some examples, a universal forwarder (e.g., a Splunk™ universal forwarder). The universal forwarder may be cross-compatible with various types of hardware, operating systems, etc. The universal forwarder may be an executable (e.g., a software instance) on the support device 104. The universal forwarder may collect and forward (e.g., send) the raw utilization data 111 (e.g., machine-generated raw data) to the monitoring and analytics system 110 and/or the database 108. As described herein, the database 108 may receive raw utilization data 111 associated with an instrument device 102 and generate structured utilization data 109 based on the raw utilization data 111 corresponding to that support device 104. In some examples, the forwarding component 105 may generate (e.g., via the universal forwarder) the structured utilization data 109 based on the raw utilization data 111.

The forwarding components 105 of the supporting devices 104 may facilitate improved methods for collecting and analyzing instrument-related data or information and support device-related data or information. For example, as noted herein, collecting and analyzing data from a multitude of instrument devices 102 may present issues related to scalability of the monitoring and analytics system 110 due to the instrument devices 102 each having their own proprietary data format (e.g., vendor-specific formats). Using the raw utilization data 111 and the corresponding structured utilization data 109 as a proxy for instrument utilization, the monitoring and analytics system 110 may circumvent computational, and possibly fiscal, ramifications of relying solely on instrument-specific utilization data. The monitoring and analytics system 110 may thus be considered “instrument agnostic” in the sense that utilization of the instrument devices 102, regardless of type/manufacturer, may be determined.

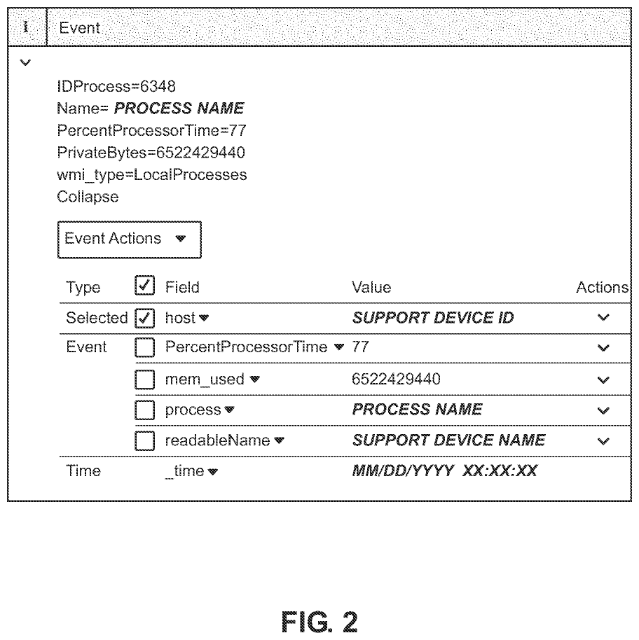

FIG. 2 shows example raw utilization data 111 for an example support device 104. The example raw utilization data 111 shown in FIG. 2 may correspond to a time, or range or time, during which the example support device 104 was receiving and/or processing input data collected during studies, process-oriented workflows and/or experiments by the corresponding instrument device 102. As can be seen in FIG. 2, the raw utilization data 111 for the example support device 104 includes a variety of information. Some of that information (e.g., portions of the raw utilization data 111) may be used as a proxy for instrument utilization for the corresponding instrument device 102.

For example, the field “PercentProcessorTime” may indicate an amount of processor(s) usage for the example support device 104. As another example, the field “mem_used” may indicate an amount of memory (e.g., RAM) usage for the example support device 104. The example raw utilization data 111 shown in FIG. 2 may identify the example support device 104 by one or more fields, such as the “host” field and the “readableName” field. The example raw utilization data 111 shown in FIG. 2 may also identify a process(es) that resulted in the raw utilization data 111 (e.g., an event name) using the “process” field. The raw utilization data 111 shown in FIG. 2 may also identify a time that corresponds to the process(es) that resulted in the raw utilization data 111 using the “_time” field (e.g., a timestamp). The format of the raw utilization data 111 shown in FIG. 2 is an example only and not meant to be restrictive. The support devices 104 may provide different formats of raw utilization data 111 that may be used by the monitoring and analytics system 110.

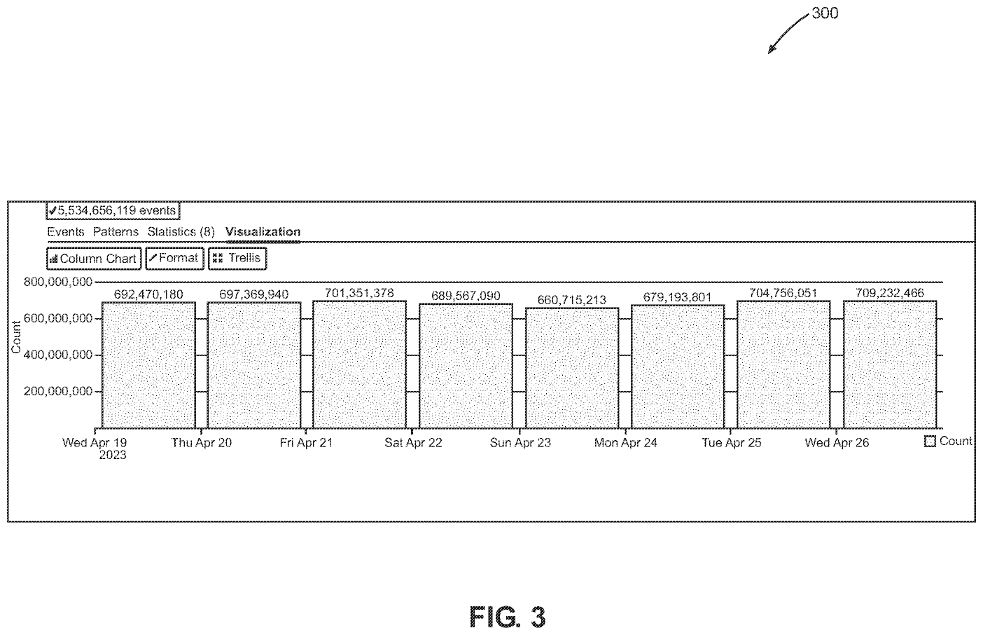

Each of the support devices 104 may send raw utilization data 111 for numerous events, processes, etc., related to the instrument devices 102 throughout a given period of time. For example, as shown in dashboard 300 of FIG. 3., raw utilization data 111 for nearly 680 million events/processes may be ingested by the monitoring and analytics system 110 in just one day.

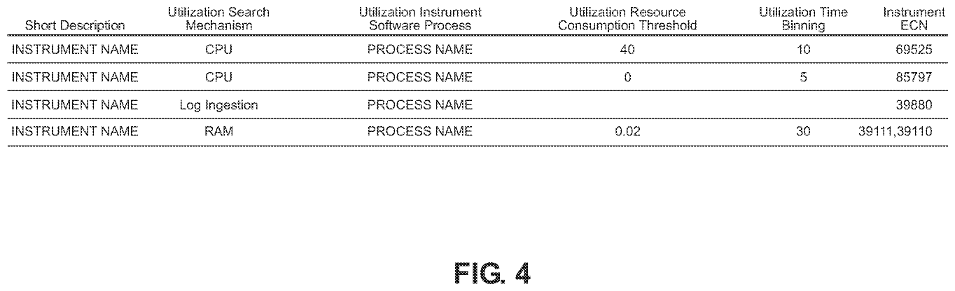

As described herein, the database 108 may receive raw utilization data 111 associated with an instrument device 102 and generate structured utilization data 109. FIG. 4 shows an example of such structured utilization data 109 for four example support devices 104 based on corresponding raw utilization data 111. As shown in FIG. 4, the structured utilization data 109 may comprise aspects or portions of raw utilization data 111 organized according to a database schema. To facilitate querying of the structured utilization data 109 via the monitoring and analytics system 110, the database 108 may be configured to organize the raw utilization data 111 into a plurality of fields, such as: “Name,” “Utilization Search Mechanism,” “Utilization Instrument Software Process,” “Utilization Resource Consumption Threshold,” “Utilization Time Binning,” and “Instrument ECN.” The “Name” field in the structured utilization data 109 may correspond to the “host” field shown in the raw utilization data 111 in FIG. 2. The “Instrument ECN” field in the structured utilization data 109 may be an identifier for the particular instrument device 102 corresponding to the support device 104.

The database 108 (or another device of the system 100-not shown) may be configured to modify a standard (or existing) format for configuration data so it may be used when generating the structured utilization data 109. For example, the structured utilization data 109 may be based on, or comprise, modified configuration data related to the support devices 104 and the instrument devices 102. The configuration data may correspond to a Configuration Management Database (“CMDB”) already existing in the database 108 (or another device of the system 100—not shown). The CMDB may store a catalog of information related to which particular instrument device 102 is in communication with a particular support device 104. Other information may be provided in the CMDB, and the fields within the CMDB may be modified to include the “Utilization Search Mechanism” field, the “Utilization Instrument Software Process” field, the “Utilization Resource Consumption Threshold” field, and the “Utilization Time Binning” field shown in FIG. 4. Those fields may be added to a database schema for the configuration data in the CMDB (the “CMDB schema”) to facilitate generation of the structured utilization data 109 as well as to facilitate queries performed by via the monitoring and analytics system 110 as further described herein.

For example, the “Utilization Search Mechanism” field may be added to the CMDB schema so that the value for “PercentProcessorTime” and/or “mem_used” within the raw utilization data 111 for a particular support device 104 may be queried to determine processor usage and/or memory usage, respectively. As another example, the “Utilization Instrument Software Process” field may be added to the CMDB schema so that the value for the “process” field within the raw utilization data 111 for the particular support device 104 may be queried to determine the particular process(es) the support device 104 was executing/running that resulted in the raw utilization data 111 (e.g., an event(s) name). As a further example, the “Utilization Resource Consumption Threshold” field may be added to the CMDB schema so that the values for “PercentProcessorTime” and/or “mem_used” within the raw utilization data 111 for the support device 104 may be used as a filtering mechanism, depending on a corresponding value for each and a configurable threshold value for each as further described herein. The “Utilization Time Binning” field may be added to the CMDB schema so that the value for the “_time” field (e.g., a timestamp) within the raw utilization data 111 for the support device 104 may be used to bin/group the utilization data within a period(s) of time (e.g., to group the data within “bins” of time).

The monitoring and analytics system 110 may query the structured utilization data 109 and/or the raw utilization data 111 stored in the database 108. For example, the monitoring and analytics system 110 may query the structured utilization data 109 and/or the raw utilization data 111 stored in the database 108 according to the modified CMDB schema described herein (e.g., comprising the “Name,” “Utilization Search Mechanism,” “Utilization Instrument Software Process,” “Utilization Resource Consumption Threshold,” “Utilization Time Binning,” and/or “Instrument ECN” fields described herein). The monitoring and analytics system 110 may query the structured utilization data 109 and/or the raw utilization data 111 to determine a current or historical utilization rate for a particular instrument device 102/support device 104.

The queries may be configured to determine, among other things, (1) when the corresponding support device application 107 was executing on the corresponding support device 104 and (2) when the corresponding support device application 107 was actually consuming resources of the support device 104. By configuring the queries in this way, the monitoring and analytics system 110 may essentially filter-out false or misleading query results. For example, if the queries were not configured in this manner, a false or misleading query result may occur in situations where the support device 104 is powered on and running but the support device application 107 is not executing (e.g., the instrument device 102 is not being used). As another example, if the queries were not configured in this manner, a false or misleading query result may occur in situations where the support device 104 is powered on and running and the support device application 107 is executing but the instrument device 102 is idle.

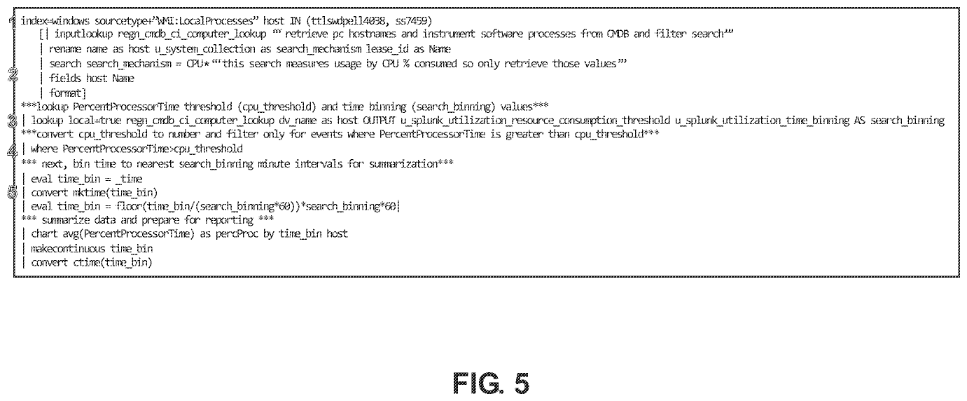

FIG. 5 shows another example of a Processor-based Utilization Query with annotations 1-5, which are included to describe various aspects/sub-queries of the Processor-based Utilization Query:
1. Query of the ingested raw utilization data 111;
2. Query of the structured utilization data 109, which is configured to return only the support devices 104 that have a search mechanism for a processor-related field (“PercentProcessorTime”) with a filter to return only the support device application 107 processes for those particular support devices 104;
3. Query of the structured utilization data 109 a second time to determine whether the “PercentProcessorTime” field meets or exceeds a configurable threshold value (“cpu_threshold”) within a time frame/bin/period (“time_bin”);
4. Filter data where the configurable threshold value was not met or exceeded; and
1. Bin the results to a set time period.

The results of the example Processor-based Utilization Query identify when the support device application 107 (and/or the instrument application 103) was running/executing and when resources being used met or exceeded the configurable threshold value (e.g., the value assigned for “cpu_threshold”). The Processor-based Utilization Query has the ability to filter down to a specific process of the support device application 107 for a specific support device 104 with a configurable threshold value for processor usage and as well as a time bin. The configurable threshold value for the Processor-based Utilization Query may be set, for example as shown in FIG. 4, at “40” to account for an amount/portion of processor(s) resources that the particular support device 104 may consume when idle and/or when processing data unrelated to an experiment or study (e.g., performing a system or software update, etc.). That is, the configurable threshold value for the Processor-based Utilization Query may function as a filter so that processor usage below the configurable threshold value is not returned as a result of the query.

As can be seen from FIG. 5, the Processor-based Utilization Query retrieves a value for comparing against the configurable threshold value using the “PercentProcessorTime” field, which may be retrieved from the corresponding raw utilization data 111 described herein. As can also be seen from FIG. 5, the time bin value may be retrieved from the raw utilization data 111 using the “_time” field (e.g., a timestamp). Using reference values rather than hardcoded values for these portions of the Processor-based Utilization Query may have significant scaling and support benefits. For example, instead of creating a unique query for each process, the monitoring and analytics system 110 may use more widely-applicable queries that function across nearly all software and hardware types—so long as processor usage may be ascertained from the corresponding raw utilization data 111 and/or structured utilization data 109.

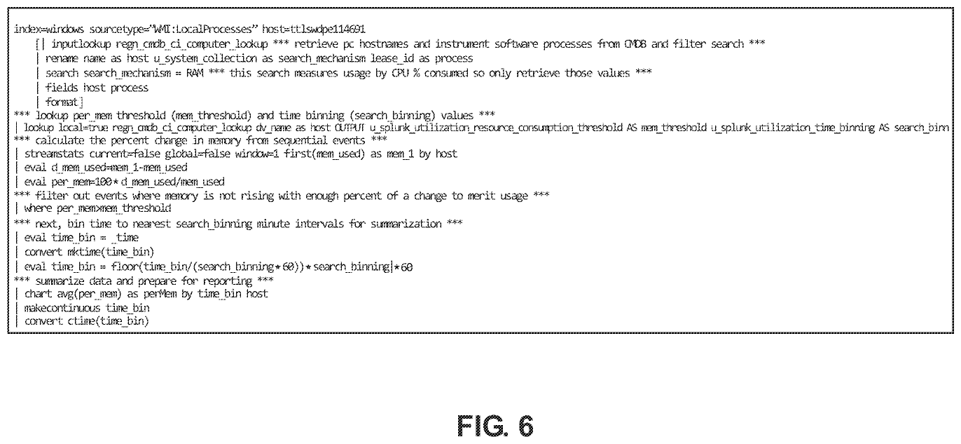

FIG. 6 shows an example query that is similar to the Processor-based Utilization Query, except that it provides results associated with memory usage of the support device 104, referred to herein as a “Memory-based Utilization Query.” The Memory-based Utilization Query may be used by the monitoring and analytics system 110 in situations where the Processor-based Utilization Query may not provide an accurate representation of actual usage of the instrument device 102 and/or the instrument application 103. For example, some processes of the instrument device 102 and/or the instrument application 103, as well as some versions/types of instrument devices 102 and/or the instrument application 103, consume very little processor resources such that the Processor-based Utilization Query may not provide an accurate representation of actual usage of the instrument device 102 and/or the instrument application 103.

As shown in FIG. 6, the Memory-based Utilization Query may share many key pieces with the Processor-based Utilization Query. However, rather than querying for memory usage above a configurable threshold (e.g., in contrast to the Processor-based Utilization Query's use of a processor usage threshold), the Memory-based Utilization Query may be configured to determine a rate of change for memory usage. For example, the rate of change for memory usage may be an indication of a percentage-based change of memory usage for the support device 104 between a first time and a second time (e.g., a rate of change during a configurable time period). The Memory-based Utilization Query may be configured to determine whether the rate of change for memory usage meets or exceeds a configurable threshold value for a result to be returned (e.g., for a result other than “null” to be returned). The Memory-based Utilization Query may use a “mem threshold” field for the configurable threshold value, which is shown in FIG. 6. The Memory-based Utilization Query may also use the “mem_used” field described above, which may be retrieved from raw utilization data 111 as shown in FIG. 2, and compared against the value assigned to the configurable threshold value (e.g., the value for the “mem_threshold” field) to determine whether the rate of change for memory usage meets or exceeds the configurable threshold value. The configurable threshold value for the Memory-based Utilization Query may be set, for example as shown in FIG. 4, at “0.2” to account for an amount/portion of memory that the particular support device 104 may consume when idle and/or when processing data unrelated to an experiment or study (e.g., performing a system or software update, etc.). That is, the configurable threshold value for the Memory-based Utilization Query may function as a filter so that memory usage below the configurable threshold value is not returned as a result of the query.

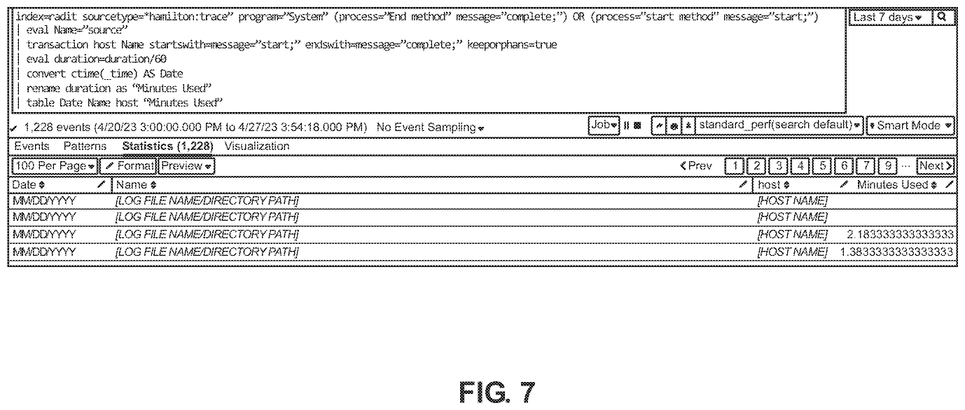

The monitoring and analytics system 110 may also use, in some examples, instrument-specific logs and/or instrument-management software information. For example, in situations where processor usage and/or memory usage of the support devices 104 is not available, the monitoring and analytics system 110 may use instrument-specific logs. Such instrument-specific logs may be used as a failover option. The instrument-specific logs may, as an example, be sent for storage in the database 108 (or another database(s)—not shown) by the instrument application 103 via the forwarding component 105. An example of an instrument-specific log is shown in FIG. 7.

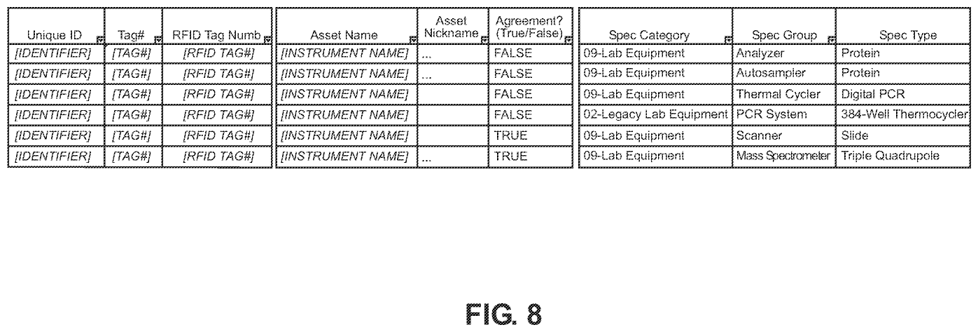

The instrument-management software information may be provided by a third-party software suite, such as Tririga™. Each of the instrument devices 102 may be identified in the instrument-management software information using an Asset tag with a unique identifier number, as shown in FIG. 8. The monitoring and analytics system 110 may store the instrument-management software information in the database 108 (or another database(s)—not shown). The monitoring and analytics system 110 may identify which support device 104 is in communication with a particular instrument device 102 by matching the Asset tag/unique identifier number for the instrument device 102 with the value in the “Instrument ECN” field in the structured utilization data 109 for the instrument device 102. As shown in FIG. 8, the instrument-management software information may include a model of the corresponding instrument device 102 as well as a field called “spec group” that identifies the type of instrument device 102 (e.g., flow cytometer, mass spectrometer, etc.).

Returning briefly to FIG. 1, the monitoring and analytics system 110 may comprise an analytics schema 113 and a dashboard module 115. The analytics schema 113 may be based on and/or comprise the raw utilization data 111, the structured utilization data 109, the instrument-specific logs, and/or the instrument-management software information described herein. For example, the analytics schema 113 may be based on, or comprise, the modified CMDB schema described herein. A dashboard module 115 of the monitoring and analytics system 110, such as Microsoft Power BI™, may receive the raw utilization data 111, the structured utilization data 109, the instrument-specific logs, and/or the instrument-management software information via automated scripting software, such as Microsoft Power Automate Scripts™. For example, the automated scripting software may provide the aforementioned data and information to the dashboard module 115 via a series of messages, such as emails, API pulls, etc.

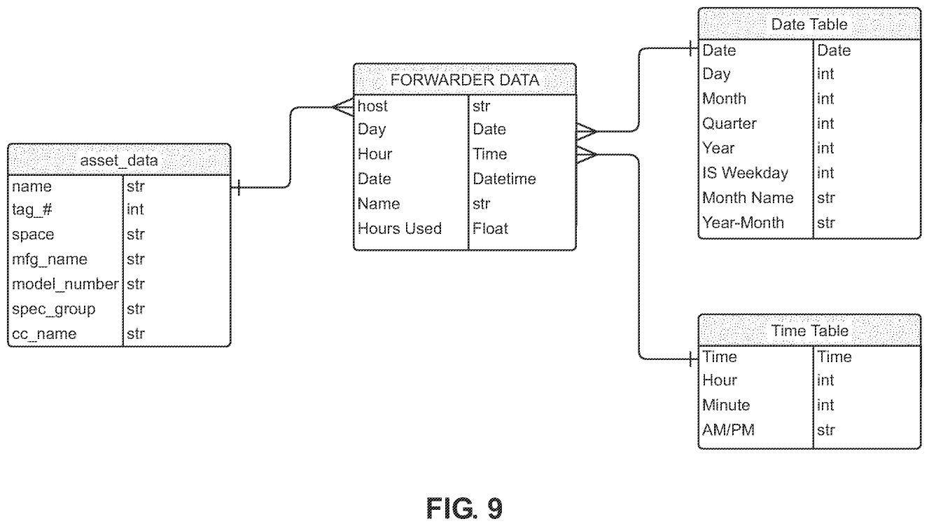

FIG. 9 shows an example of the analytics schema 113. The dashboard module 115 may retrieve the raw utilization data 111, the structured utilization data 109, the instrument-specific logs, and/or the instrument-management software information from the database 108 (and/or another database(s)—not shown) and generate the analytics schema 113. The dashboard module 115 may retrieve the underlying values for each item of the analytics schema 113 on a regular/scheduled basis (e.g., daily).

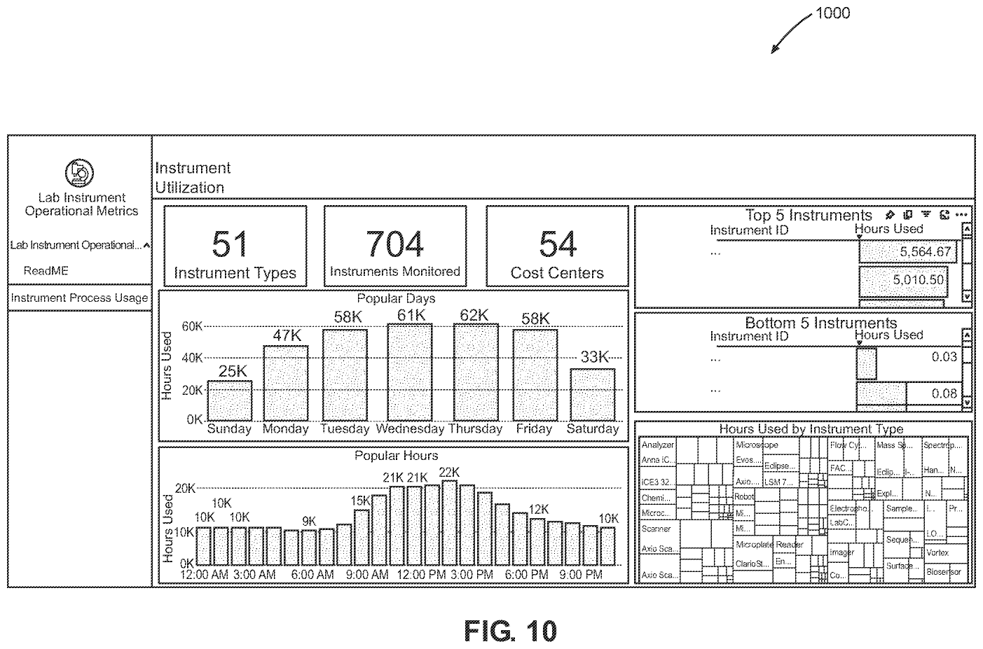

FIG. 10 shows an example dashboard 1000 (e.g., a user interface) as well as example visualizations that may be generated by the dashboard module 115 and output at one or more client devices in communication with the monitoring and analytics system 110 (e.g., computers, tablets, mobile devices, etc.).

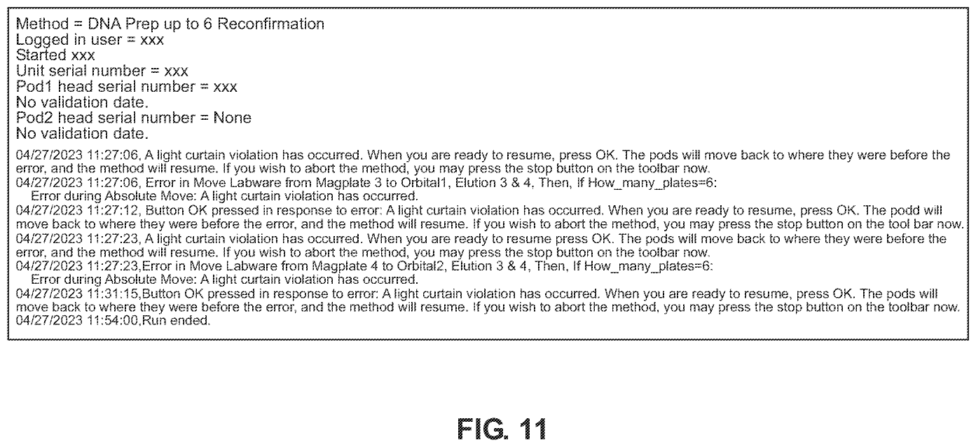

As described herein, the monitoring and analytics system 110 may use the raw utilization data 111 and the corresponding structured utilization data 109 associated with a particular instrument device 102 and/or a particular support device 104 as a proxy for instrument-specific utilization data. Such instrument-specific utilization data may be generated by the instrument application 103 of the particular instrument device 102 and/or by the support device application 107 of the particular support device 104. FIG. 11 shows an example of the instrument-specific utilization data, which may be output as one or more verbose logs.

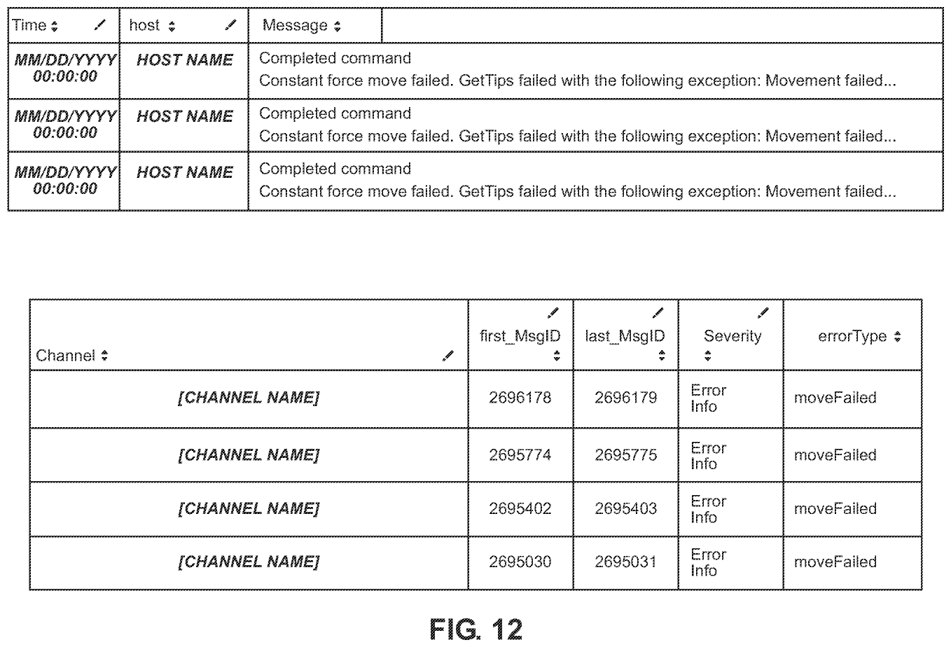

These verbose logs may contain, or be indicative of, several instrument-specific utilization metrics, such as when an instrument device 102 began and/or completed use (e.g., during an experiment or study), one or more warnings, errors, informational events, a combination thereof and/or the like. For example, FIG. 12 shows example results of a query of instrument-specific utilization data with instrument-specific filters. The particular instrument-specific filters may be dependent on the type, manufacturer, etc. of instrument device 102, a type of experiment or study being conducted, a type, developer, etc. of the instrument application 103, a combination thereof, and/or the like. The instrument-specific filters may be configured such that the query results only include relevant errors that may impact an availability of the particular instrument device 102. For example, as shown in FIG. 12, the results of querying the instrument-specific utilization data with instrument-specific filters may include a “host” field to identify the particular instrument device 102; an error “message” field to describe the error; an “errorType” to identify the type of error, etc.

As described herein, the raw utilization data 111 and the corresponding structured utilization data 109 associated with a particular instrument device 102 and/or a particular support device 104 may be used as a proxy for instrument-specific utilization data. However, using the raw utilization data 111 and the corresponding structured utilization data 109 as a proxy for instrument-specific utilization data may require deriving values for various metrics and elements that provide more information beyond identifying whether a particular instrument device 102 and/or support device 104 is “available,” “online,” etc. (e.g., available for use). A relative “health” or availability of the instrument devices 102 and/or support devices 104 may be easily determined using the instrument-specific utilization metrics within, or indicated by, the verbose logs/instrument-specific utilization data.

The instrument-specific utilization data for a particular instrument device 102 may be stored locally on the corresponding support device 104. The forwarding component 105 of the support device 104 may be configured (e.g., via the support device application 107) to send the instrument-specific utilization data (e.g., the verbose logs) to the database 108 and/or another database(s) (not shown). The instrument-specific utilization metrics described above may be extracted from the verbose logs and organized into fields that are easily readable and searchable by the monitoring and analytics system 110. The fields extracted from the verbose logs may be used to provide a “health dashboard” indicating the relative “health” or availability of instrument devices 102 and/or support devices 104.

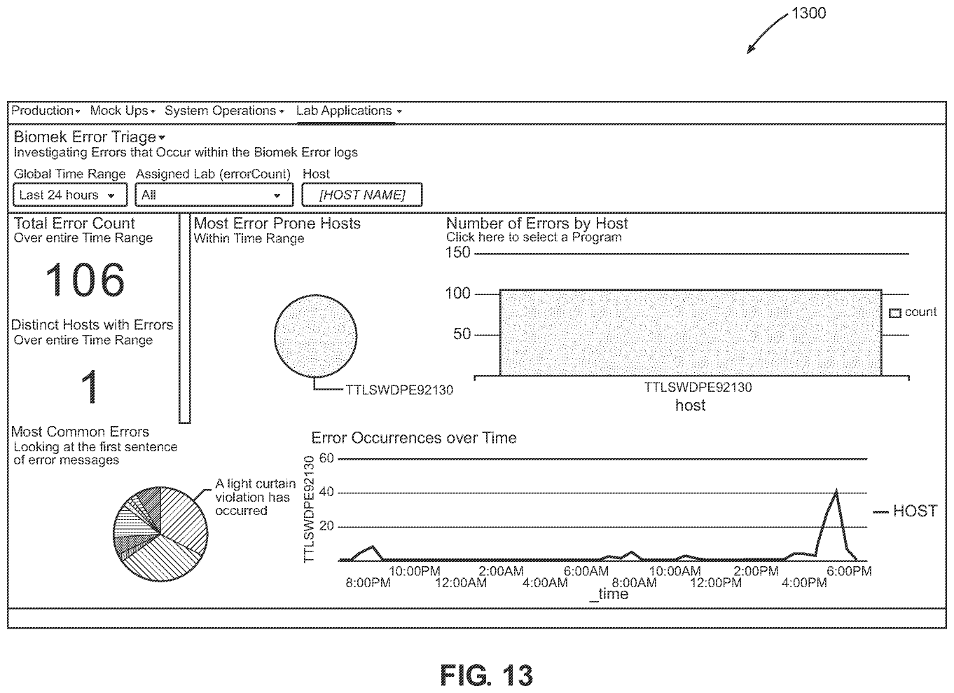

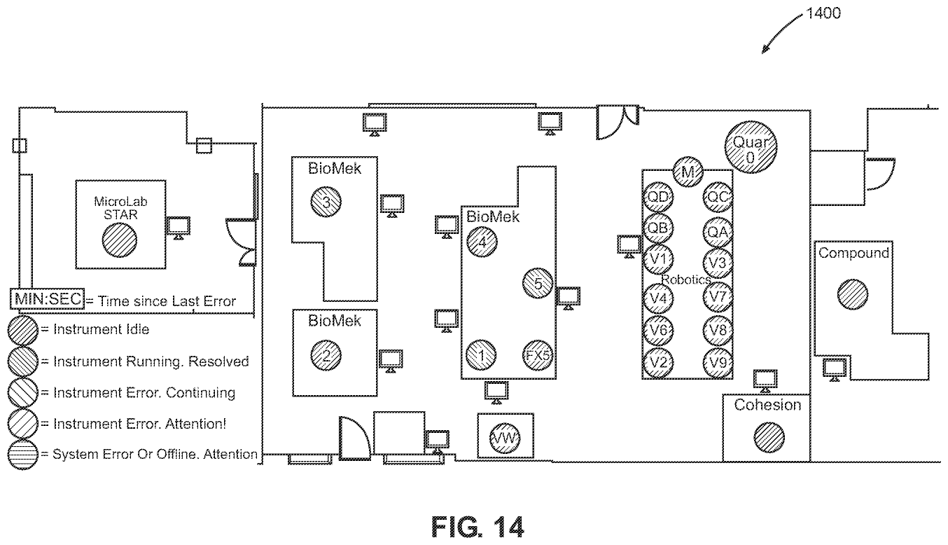

FIG. 13 shows an example health dashboard (e.g., a user interface) that the dashboard module 115 may output based on results of queries using the fields extracted from the verbose logs. FIG. 14 shows another example health dashboard with a representation of the location of each instrument device 102 such as a map/diagram of example rooms (e.g., laboratory rooms) that may house one or more of the example instrument devices 102 and support devices 104 described herein. Each circle shown on the health dashboard in FIG. 14 may correspond to a support device 104 and/or an instrument device 102. The color of a particular circle may indicate a current status of the corresponding instrument device 102 for that support device 104, such as gray for idle; green for running; yellow for a less-than-critical instrument device 102 error, red for a critical instrument device 102 error, and purple for an offline or unavailable instrument device 102. When a particular instrument device 102 is determined to be in an error state and/or otherwise not fully available for use, the monitoring and analytics system 110 may output a message via the health dashboard, which may be based on results of querying corresponding instrument-specific utilization data for a particular instrument device 102 using the instrument-specific filters described above.

The dashboard module 115 may query the verbose logs based on a timer, a schedule, a trigger based on a particular instrument-specific utilization metric, a combination thereof, and/or the like. Additionally, in some examples as shown in FIG. 14, a number may be included in the health dashboard proximate to each depicted support device 104 (e.g., within the circle) to indicate an amount of time (e.g., minutes, seconds, etc.) since the last error related to the corresponding instrument device 102 occurred. The number may be only visible when an error occurs and it may only be present until the error is cleared out, resolved, etc.

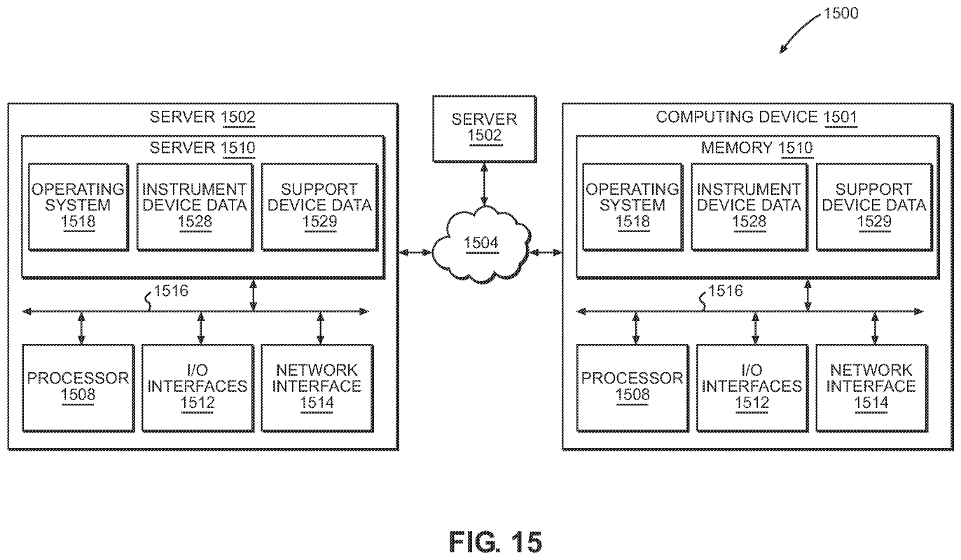

The present methods and systems may be computer-implemented. FIG. 15 shows a block diagram depicting a system/environment 1500 comprising non-limiting examples of a computing device 1501 and a server 1502 connected through a network 1504. Any of the entities or devices of the system 100 may be a computing device. In an aspect, some or all steps of any described method may be performed on a computing device as described herein. The computing device 1501 may comprise one or multiple computers configured to support device data 1529 and/or the like. The server 1502 may comprise one or multiple computers configured to store instrument device data 1528. Multiple servers 1502 may communicate with the computing device 1501 via the through the network 1504.

The computing device 1501 and the server 1502 may be a computer that, in terms of hardware architecture, generally includes a processor 1508, system memory 1510, input/output (I/O) interfaces 1512, and network interfaces 1514. These components (1508, 1510, 1512, and 1514) are communicatively coupled via a local interface 1516. The local interface 1516 may be, for example, but not limited to, one or more buses or other wired or wireless connections, as is known in the art. The local interface 1516 may have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 1508 may be a hardware device for executing software, particularly that stored in system memory 1510. The processor 1508 may be any custom made or commercially available processor, a central processing unit (CPU), an auxiliary processor among several processors associated with the computing device 1501 and the server 1502, a semiconductor-based microprocessor (in the form of a microchip or chip set), or generally any device for executing software instructions. When the computing device 1501 and/or the server 1502 is in operation, the processor 1508 may execute software stored within the system memory 1510, to communicate data to and from the system memory 1510, and to generally control operations of the computing device 1501 and the server 1502 pursuant to the software.

The I/O interfaces 1512 may be used to receive user input from, and/or for providing system output to, one or more devices or components. User input may be provided via, for example, a keyboard and/or a mouse. System output may be provided via a display device and a printer (not shown). I/O interfaces 1512 may include, for example, a serial port, a parallel port, a Small Computer System Interface (SCSI), an infrared (IR) interface, a radio frequency (RF) interface, and/or a universal serial bus (USB) interface.

The network interface 1514 may be used to transmit and receive from the computing device 1501 and/or the server 1502 on the network 1504. The network interface 1514 may include, for example, a 10BaseT Ethernet Adaptor, a 10BaseT Ethernet Adaptor, a LAN PHY Ethernet Adaptor, a Token Ring Adaptor, a wireless network adapter (e.g., WiFi, cellular, satellite), or any other suitable network interface device. The network interface 1514 may include address, control, and/or data connections to enable appropriate communications on the network 1504.

The system memory 1510 may include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the system memory 1510 may incorporate electronic, magnetic, optical, and/or other types of storage media. Note that the system memory 1510 may have a distributed architecture, where various components are situated remote from one another, but may be accessed by the processor 1508.

The software in system memory 1510 may include one or more software programs, each of which comprises an ordered listing of executable instructions for implementing logical functions. In the example of FIG. 15, the software in the system memory 1510 of the computing device 1501 may comprise the support device data 1529, the instrument device data 1528, and a suitable operating system (O/S) 1518. In the example of FIG. 15, the software in the system memory 1510 of the server 1502 may comprise the support device data 1529, the instrument device data 1528, and a suitable operating system (O/S) 1518. The operating system 1518 essentially controls the execution of other computer programs and provides scheduling, input-output control, file and data management, memory management, and communication control and related services.

For purposes of illustration, application programs and other executable program components such as the operating system 1518 are shown herein as discrete blocks, although it is recognized that such programs and components may reside at various times in different storage components of the computing device 1501 and/or the server 1502. An implementation of the system/environment 1500 may be stored on or transmitted across some form of computer readable media. Any of the disclosed methods may be performed by computer readable instructions embodied on computer readable media. Computer readable media may be any available media that may be accessed by a computer. By way of example and not meant to be limiting, computer readable media may comprise “computer storage media” and “communications media.” “Computer storage media” may comprise volatile and non-volatile, removable and non-removable media implemented in any methods or technology for storage of information such as computer readable instructions, data structures, program modules, or other data. Exemplary computer storage media may comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer.

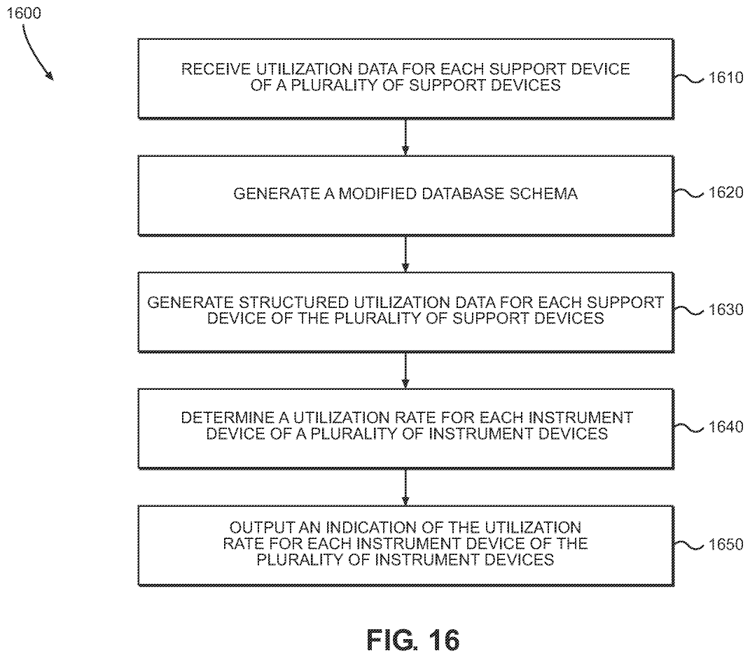

FIG. 16 shows a flowchart of an example method 1600 for improved instrument monitoring and analytics. The method 1600 may be performed in whole or in part by a single computing device, a plurality of computing devices, and the like. For example, the steps of the method 1600 may be performed by a computing device within or associated with the monitoring and analytics system 110 shown in FIG. 1 and/or a computing device in communication with any of the devices/entities of the system 100.

At step 1610, a computing device may receive utilization data (e.g., raw utilization data 111) for each support device of a plurality of support devices. Each support device of the plurality of support devices may be in communication with (e.g., communicably coupled to) at least one instrument device of a plurality of instrument devices. The plurality of support devices may receive input data from the plurality of instrument devices (e.g., the “input data” described herein).

The utilization data and/or the structured utilization data for each support device of the plurality of support devices may be based on the input data received from the plurality of instrument devices. For example, the utilization data and/or the structured utilization data may be associated with execution of at least one instrument application executing on each instrument device of the plurality of instrument devices. That is, the utilization data and/or the structured utilization data for each support device may be associated with that support device's control/operation of the corresponding instrument device as it collects and/or generates the corresponding input data. For example, the utilization data and/or the structured utilization data for each support device may be associated with that support device receiving, processing, and/or sending the input data (e.g., for storage of the input data at the monitoring and analytics system 110). The input data associated with a particular instrument device may be received by the corresponding support device via the at least one instrument application executing on the particular instrument device. For example, the support device may execute at least one support device application (e.g., support device application 107) that facilitates the support device operating/controlling the instrument device and receiving the corresponding input data.

At step 1620, the computing device may generate a modified database schema (e.g., the analytics schema 113). The modified database schema may comprise a plurality of fields (e.g., the “Name,” “Utilization Search Mechanism,” “Utilization Instrument Software Process,” “Utilization Resource Consumption Threshold,” “Utilization Time Binning,” and “Instrument ECN” fields described herein). For example, a database schema for a database associated with configuration data for the plurality of instrument devices and/or the plurality of support devices (e.g., the CMDB schema as described herein) may be modified by appending the plurality of fields to the database schema. The plurality of fields may be appended to database schema to facilitate querying the utilization data associated with a specific instrument device of the plurality of instrument devices (e.g., based on the “Instrument ECN” field).

At step 1630, the computing device may generate structured utilization data for each support device of the plurality of support devices. The computing device may generate the structured utilization data for each support device of the plurality of support devices based on the utilization data for each support device of the plurality of support devices and the modified database schema. For example, the structured utilization data may comprise the plurality of fields. The structured utilization data may be generated, in some examples, by a support device(s). For example, a first support device of the plurality of support devices may generate the structured utilization data associated with the first support device based on the utilization data associated with the first support device and/or the modified database schema. Additionally, or in the alternative, the database associated with the plurality of support devices may generate the structured utilization data based on the utilization data associated with each support device of the plurality of support devices and/or the modified database schema.

At step 1640, the computing device may determine a utilization rate for each instrument device of the plurality of instrument devices. For example, the computing device may determine the utilization rate for each instrument device based on at least one query associated with the structured utilization data for each support device of the plurality of support devices. The at least one query may comprise at least one field of the plurality of fields (e.g., the “Name,” “Utilization Search Mechanism,” “Utilization Instrument Software Process,” “Utilization Resource Consumption Threshold,” “Utilization Time Binning,” and/or “Instrument ECN” field described herein).

The at least one field may be associated with, or indicative of, processor usage of each support device of the plurality of support devices. Determining the utilization rate for each instrument device of the plurality of instrument devices may comprise determining that the processor usage, indicated by or associated with at least one field, meets or exceeds a configurable threshold value. The at least one field within the structured utilization data for each support device may be indicative of the processor usage for the corresponding support device. The configurable threshold value may be based on a processor usage associated with an idle state of the support device. Other examples are possible as well.

Additionally, or in the alternative, the at least one field may be associated with, or indicative of, memory usage of each support device of the plurality of support devices. Determining the utilization rate for each instrument device of the plurality of instrument devices may comprise determining that the memory usage, indicated by or associated with at least one field, meets or exceeds a configurable threshold value. The at least one field within the structured utilization data for each support device may be indicative of the memory usage for the corresponding support device. The configurable threshold value may be based on a memory usage associated with an idle state of the support device. At step 1650, the computing device may output an indication of the utilization rate for each instrument device of the plurality of instrument devices. For example, the computing device may output an indication of the utilization rate for each instrument via a graphical user interface, such as one or more of the dashboards described herein.

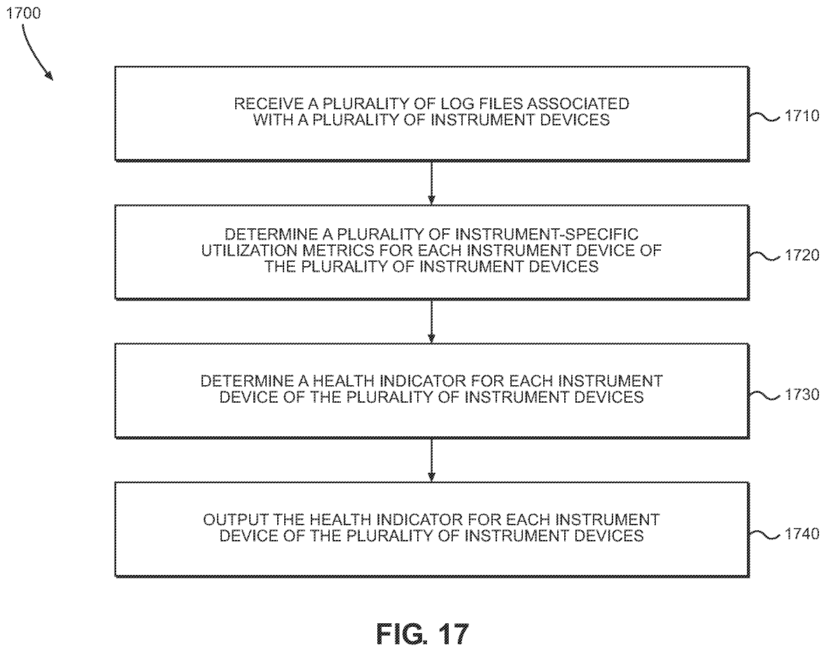

FIG. 17 shows a flowchart of an example method 1700 for improved instrument monitoring and analytics. The method 1700 may be performed in whole or in part by a single computing device, a plurality of computing devices, and the like. For example, the steps of the method 1700 may be performed by a computing device within or associated with the monitoring and analytics system 110 shown in FIG. 1 and/or a computing device in communication with any of the devices/entities of the system 100.

At step 1710, a computing device may receive a plurality of log files associated with a plurality of instrument devices. The computing device may receive the plurality of log files from a plurality of support device. Each support device of the plurality of support devices may be in communication with (e.g., communicably coupled to) at least one instrument device of the plurality of instrument devices. Each support device of the plurality of support devices may receive input data from the at least one instrument device corresponding to that support device. Each instrument device may execute at least one instrument application (e.g., the instrument application(s) 103). For example, each support device may operate/control the at least one instrument device to which that support device is communicably coupled via the at least one instrument application executing on that at least one instrument device. The input data associated with the at least one instrument device corresponding to a particular support device may be received via the at least one instrument application. For example, the support device may execute at least one support device application (e.g., support device application 107) that facilitates the support device operating/controlling the at least one instrument device and receiving the corresponding input data.

The plurality of support devices may receive the plurality of log files from the plurality of instrument devices (e.g., via the at least one instrument application and the at least one support device application). The plurality of log files may comprise, or be based on, a plurality of filtered verbose log files, which may be filtered to remove irrelevant information. For example, the plurality of filtered verbose log files may be based on a plurality of instrument-specific filters applied to the plurality of log files. The plurality of instrument-specific filters may comprise an instrument type, an instrument manufacturer, a type of experiment or study, a type of an instrument application, or a developer of the instrument application.

At step 1720, the computing device may determine a plurality of instrument-specific utilization metrics for each instrument device of the plurality of instrument devices. For example, the computing device may determine the plurality of instrument-specific utilization metrics based on the plurality of log files. In some examples, the plurality of support devices may send the plurality of log files to a monitoring and analytics system (e.g., the monitoring and analytics system 110), and the plurality of instrument-specific utilization metrics may be generated by the monitoring and analytics system. The instrument-specific utilization metrics may indicate a number of states for the corresponding instrument device. For example, the instrument-specific utilization metrics for a first instrument device of the plurality of instrument devices may comprise at least one of: an indication of a starting use time for the first instrument device, an indication of an ending use time for the first instrument device, an indication of one or more warnings associated with the first instrument device, an indication of one or more errors associated with the first instrument device, or an indication of one or more informational events associated with the first instrument device.

At step 1730, the computing device may determine a health indicator for each instrument device of the plurality of instrument devices. For example, the computing device may determine the health indicator for each instrument device based on the plurality of instrument-specific utilization metrics for each instrument device of the plurality of instrument devices. The health indicator may be indicative of an availability of the corresponding instrument device. For example, the health indicator for each instrument device may indicate whether that instrument device is associated with one of a plurality of “availability states.” For example, the health indicator for a first instrument device of the plurality of instrument devices may indicate the first instrument device is associated with an idle state, a running state, a less-than-critical error state, a critical error state, or an offline state.

At step 1740, the computing device may output the health indicator for each instrument device of the plurality of instrument devices. For example, the computing device may output the health indicator for each instrument device of the plurality of instrument devices via a graphical user interface. The health indicator for each instrument device of the plurality of instrument devices may be indicative of an amount of time since that instrument device was associated with at least one error state. The computing device may determine when each instrument device of the plurality of instrument devices is no longer associated with the at least one error state. The computing device may output, via the graphical user interface, a refreshed health indicator for an instrument device when it is determined that the instrument device is no longer associated with the at least one error state. Other examples are possible as well.

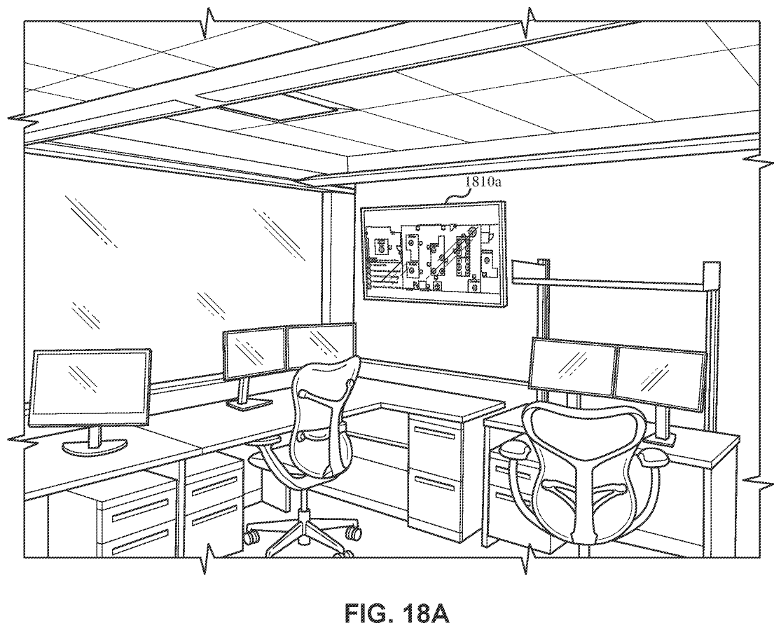

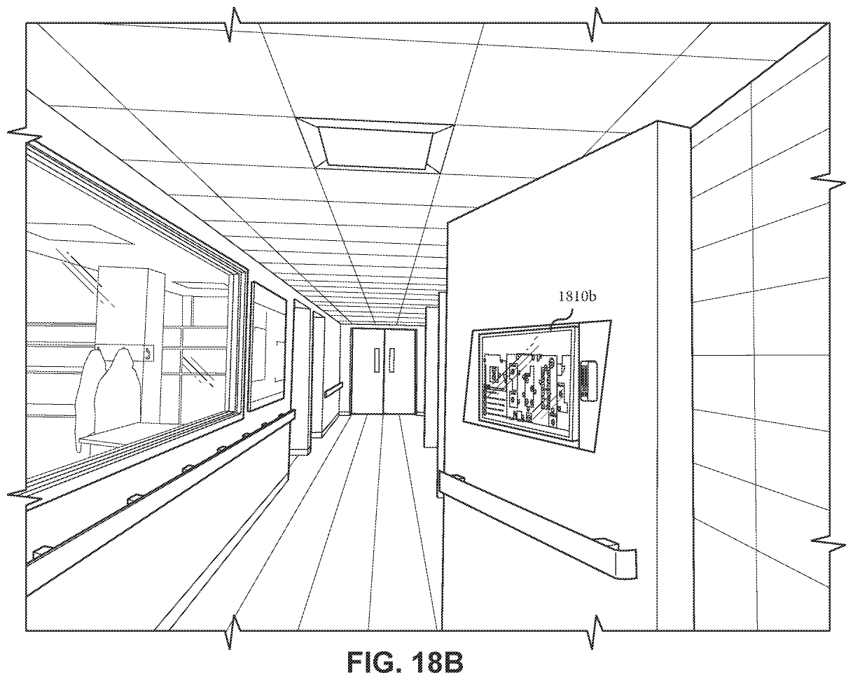

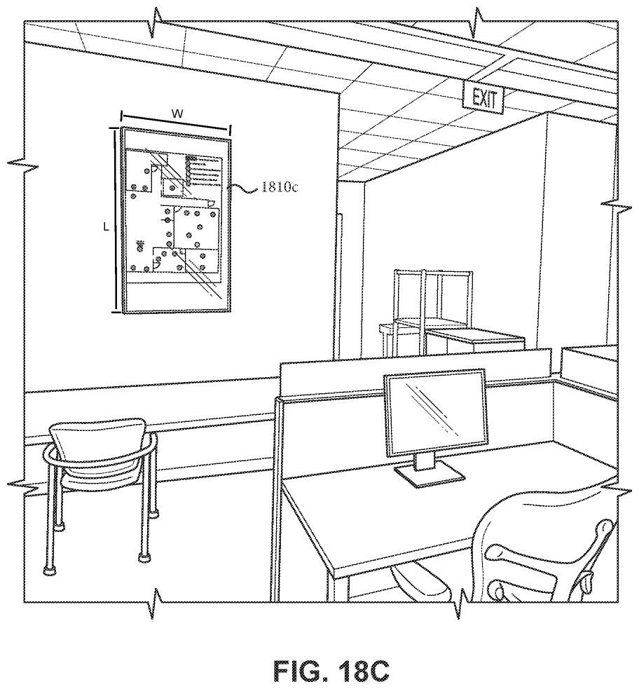

FIGS. 18A-C illustrate example information devices 1810a-c configured to present information such as dashboard 1300 or dashboard 1400 in FIGS. 13 and 14. Each information device of information devices 1810a-c may include a display, screen, touchscreen, graphical user interface, speaker, or other suitable output device. Information devices 1810a-c may be arranged and dimensioned in a suitable manner to convey visual and/or auditory information to one or more persons in an environment around information devices 1810a-c.

For example, information devices 1810a-c may be disposed in a hallway, office, laboratory, or other suitable environment. Information devices 1810a-c may have dimensions and image resolutions such that a person having 20/20 vision can clearly see information on the devices from a significant distance such as about 10 feet, about 20 feet, about 30 feet, about 40 feet, about 50 feet, or other suitable distance. Information devices 1810a-c may include one or more speakers configured to transmit auditory information such that a person having substantially normal hearing can clearly hear auditory information emitted from the devices from a significant distance such as about 10 feet, about 20 feet, about 30 feet, about 40 feet, about 50 feet, or other suitable distance.

For example, information devices 1810a-c may be planar displays such as flat screen TVs having a diagonal dimension of about 42″, about 50″, about 55″, about 60″, about 75″, about 100″, or a suitable diagonal dimension. Information devices may have a length and width (labeled L and W respectively in FIG. 18C).

A width W of information devices 1810a-c may be about 18″, about 20″, about 24″, about 25″, about 27″, about 30″, about 36″, about 40″, about 44″, about 48″, about 50″, about 55″, about 60″, or any suitable width W.

A length L of information devices 1810a-c may be about 24″, about 25″, about 27″, about 30″, about 36″, about 40″, about 44″, about 48″, about 50″, about 55″, about 60″, about 72″, about 80″, about 90″, about 100″ or any suitable length L.

In general, a given information device may have a particular size (length and width dimensions), aspect ratio, orientation (e.g., landscape or portrait) and/or placement in a given environment based at least in part on the physical floorplan of the environment, such that the display of information may be customized to the environment. For example, FIG. 18A illustrates an example of a first information device in a landscape orientation configured to present information (e.g., the various dashboards described herein) in an office environment with a physical floorplan for multiple occupancy. FIG. 18B illustrates another example of a second information device configured to present information in a common area (e.g., a hallway) connecting multiple research laboratory and/or office environments. FIG. 18C illustrates yet another example of a third information device in a portrait orientation configured to present information in a laboratory environment.

Each information device 1810a-c may be communicatively coupled to the monitoring and analytics system 110 and/or with a computing device such as a support device 104 configured to control a content of the information device such as which dashboard to display and what information to display on the dashboard. The monitoring and analytics system 110, and/or one or more computing devices may be communicatively coupled to the respective information device via one or more communications protocols such as an high-definition multimedia interface (HDMI) connection, a digital visual interface (DVI) connection, or any suitable protocol. Respective support devices 104 for information devices 1810a-c may operate support device application 107 to load a webpage, image, video, presentation, or any suitable source of information for presentation on 1810a-c.

The monitoring and analytics system 110, and/or each support device application 107, may update the contents of 1810a-c with a suitable frequency such as about once every 5 seconds, about once every 10 seconds, about once every 15 seconds, about once every 30 seconds, about once every 60 seconds, about once every two minutes, about once every three minutes, about once every five minute, about once every 10 minutes, about once every 15 minutes, about once every 20 minutes, about once every 30 minutes, about once every hour, about once every two hours, about once every four hours, about once every six hours, about once every 12 hours, about once every day, or with any suitable frequency. More generally, it should be appreciated that information relating to the contents may be pushed to (or requested/pulled by) a given information device periodically or on demand.

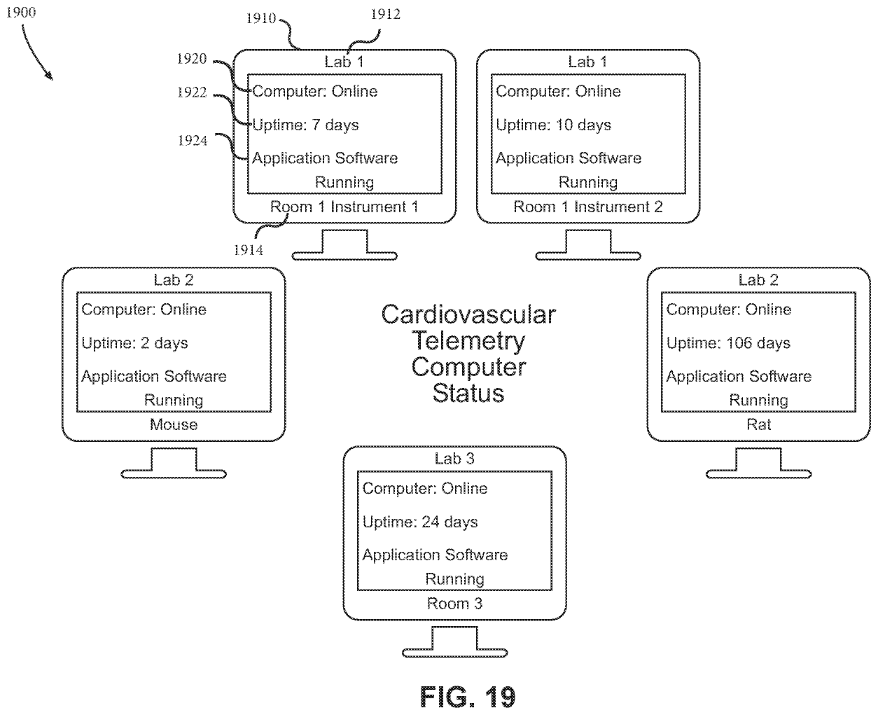

FIG. 19 illustrates a dashboard 1900 for monitoring one or more support devices 104 and providing device status updates in accordance with the present technology. While not explicitly shown in FIG. 19, it should be appreciated that in some example implementations a bounding box may be employed around a perimeter of the dashboard (and the bounding box may be scaled to the physical dimensions of a given information device on which the dashboard is displayed). Dashboard 1900 may include support device icons 1910 representing respective support devices 104 in a lab space, office, or other area. Each support device icon 1910 may include a connection status 1920, uptime 1922, and application software status 1924. Additionally, support device icon 1910 may include a location identifier 1912, and instrument identifier 1914. For example, dashboard 1900 may provide monitoring for telemetry experiments, which are in vivo experiments (e.g., collecting data from animals, including with implanted sensors) lasting days, weeks, or months. One or more support devices 104 may continuously collect data from these experiments; if these support devices 104 stop functioning properly, the interruption of data may degrade or ruin the experiment. In the worst case, animal subjects may need to be sacrificed due to the disruption of the experiment. Dashboard 1900 may provide a mechanism to quickly alert one or more persons associated with a support device 104 about interruptions to experiments, data collection, and other processes.

Dashboard module 115 may query each support device of the one or more support devices 104, for example using support device application 107. If a response is received, dashboard module 115 may determine that the respective support device is online and may indicate connection status 1920 as “Online” in the support device icon 1910 corresponding to the respective support device. Alternatively, if dashboard module 115 receives an indication from support device application 107 that a support device 104 is not properly communicating with a network, instrument, or device, or if dashboard module 115 receives no response at all from support device application 107, dashboard module 115 may indicate connection status 1920 as “Offline” and output an additional message or alert on a dashboard so that users of support device 104 are aware that a support device 104 may not be properly connected or communicating.

Monitoring and analytics system 110 and/or dashboard module 115 may transmit a message via text, email, instant messaging, or any suitable communication channel to one or more users associated with support device 104, instrument device 102, and/or an operation performed by instrument device 102 (such as an automated mixing of chemical solutions). For example, monitoring and analytics system 110 and/or dashboard module 115 may query structured utilization data 109 and assess whether one or more specific users are associated with support device 104 and/or instrument device 102. Monitoring and analytics system 110 and/or dashboard module 115 may query instrument-specific logs, verbose logs, or any other suitable log to determine one or more users associated with support device 104 and/or instrument device 102. Once the one or more users have been identified, a notification may be sent to the one or more users alerting them that support device 104 is offline, allowing them to potentially intervene in an ongoing experiment and prevent potential data loss.

Monitoring and analytics system 110 and/or dashboard module 115 may query instrument-specific logs, verbose logs, structured utilization data 109, and/or raw utilization data 111 to assess whether an experiment is occurring at a given time. For example, monitoring and analytics system 110 may parse instrument-specific logs to determine one or more periods of time during which instrument device 102 has been in continuous or regular use and may compare the usage to one or more usage patterns defining an experimental usage. If the usage indicates that an experiment is occurring or was occurring when a connection status 1920 of support device 104 changed from “Online” to “Offline” (e.g., due to a software or hardware crash, reboot, software installation, etc.), monitoring and analytics system 110 may transmit a message to one or more users associated with the experiment in order to alert them to a potential need to intervene in their experiment, backup data associated with the experiment, or request technical assistance.

Additionally or alternatively, monitoring and analytics system 110 and/or dashboard module 115 may create a service request or service ticket for support device 104 in response to detecting a problem with instrument device 102 or support device 104. For example, monitoring and analytics system 110 may create a service ticket indicating a need for a technician to assess an issue with an support device 104 when the connection status 1920 of the support device 104 changes “Online” to “Offline.” A service ticket may further indicate the existence of a particular issue, for example that a software bug within support device application 107 has caused support device 104 to crash and that a technician needs to reinstall or update support device application 107.

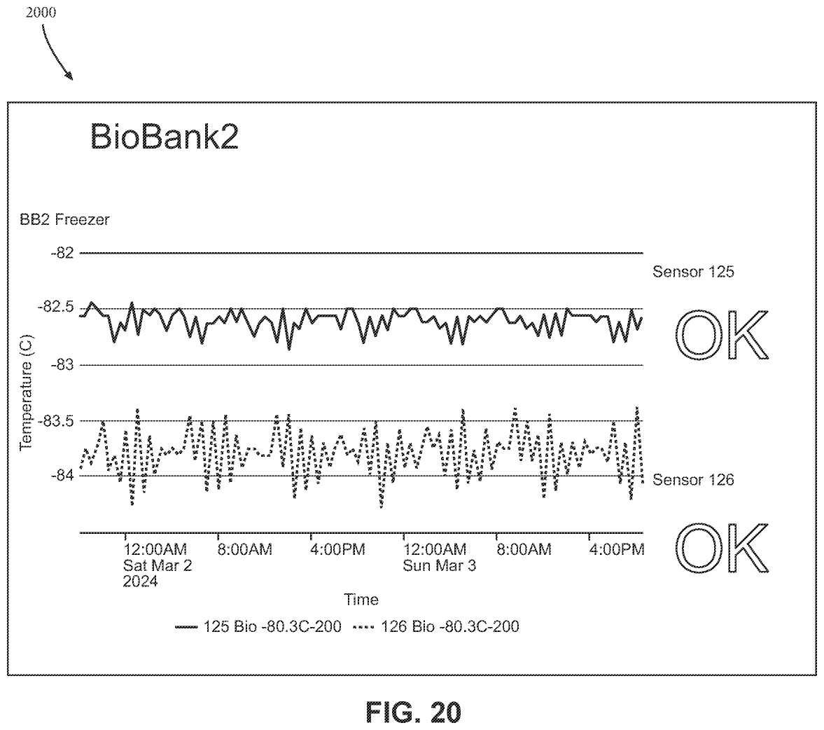

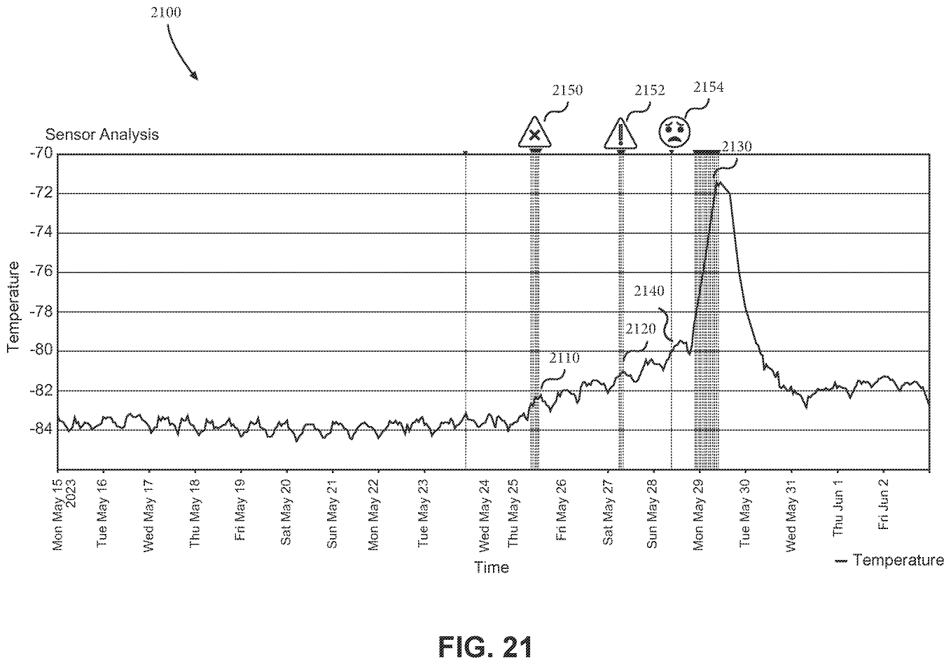

FIG. 20 and FIG. 21 illustrate predictive health monitoring dashboards for monitoring one or more instrument devices 102. FIG. 20 shows a dashboard 2000 for monitoring sensor inputs for an exemplary instrument device 102 (in this case, temperature sensors for a biological sample freezer) over time. A sensor input for an instrument device 102 may give an indication of a status for the instrument device 102, such as an operational status, a usage status, an instrument health status, or any suitable status. A sensor input for an instrument device 102 may correspond to an operational parameter of the instrument device, such as a temperature, voltage, current, frequency, pH, humidity, moisture content, fluid level, velocity, mass, weight, dimension, position, orientation, angle, rotation, acceleration, or other suitable operational parameter.

In FIG. 20, temperature sensor 125 and temperature sensor 126 may each monitor a different portion of instrument device 102. The temperature sensors may have a threshold acceptable temperature range inside of which the temperature fluctuates. For example, temperature sensor 125 may nominally fluctuate between about −82.5° C. and about −83° C. Temperature sensor 126 may nominally fluctuate between about −83.5° C. and about −84.5° C.

When instrument devices such as biological sample freezers encounter failures, users and managers of these instrument devices may not become aware of the failures until days after the failures occur. This can lead to rushed, stressful attempts to save samples contained in the failed instrument device, or in some cases leads to sample loss entirely. Dashboard 2100, optionally in conjunction with messaging and alert functionality described above, may provide an early indication of a failure or error event associated with instrument device 102 and/or adjust an operation of instrument device 102.

For example, monitoring and analytics system 110 may ingest time series temperature data for temperature sensor 125 and temperature sensor 126. Monitoring and analytics system 110 may then apply a control algorithm based on the time series temperature data and identify instrument device errors or failures before samples are damaged, thus leveraging predictive analytics for instrument device control.

To implement such a control algorithm, monitoring and analytics system 110 may calculate a target value that functions as a center line, anchor, or ideal value for an operational parameter based on sensor data or other data. For example, monitoring and analytics system 110 may calculate the target value as an average using historical data points (e.g., an average across all ingested data up to a predetermined point, a moving average such as an average of the most recent X datapoints, X periods of time [such as minutes, hours, days], etc., where X may be any suitable value such as 100, 1000, 10,000, 100,000, or the like), or a fixed target value may be chosen manually, for example by a technician, instrument user, instrument administrator, computer program such as instrument application 103 or support device application 107, or other suitable entity.

Monitoring and analytics system 110 may then calculate standard deviation threshold values representing one, two, and three standard deviations for the time series data over the chosen window. Monitoring and analytics system 110 may ingest and monitor the time series data in real time and compare each ingested value or set of values to the target value and the standard deviation threshold values and take one or more actions if one or more of the ingested values are outside of the standard deviation threshold values. A standard deviation threshold or other suitable value may be selected as a “control limit,” which may be a threshold at which human intervention is required even if the control limit is exceeded at a single data point.

Monitoring and analytics system 110 may compare each ingested data point or set of data points from the time series data to one or more rules and trigger an appropriate response to take, including adjusting an operation of instrument device 102 (i.e., any adjustable operation that is controllably performed by the instrument device). Adjusting an operation of instrument device 102 may include terminating the operation of the instrument device 102, controlling the operation of instrument device 102 to reverse an erroneous operation (e.g., to increase a flow rate of a coolant in a freezer to lower a temperature of the freezer when the temperature rises above a threshold), increasing or decreasing a physical parameter associated with the instrument device 102 (such as a temperature, voltage, current, frequency, pH, humidity, moisture content, fluid level, emitted electromagnetic radiation, or any suitable physical parameter).

Monitoring and analytics system 110 may determine, in accordance with an exemplary first rule, that any data point from an ingested time series dataset that exceeds a control limit may be defined as a large shift. Large shifts may result in monitoring and analytics system 110 transmitting a message to one or more technicians indicating that an urgent level of intervention is required (e.g., that a technician should be dispatched to examine instrument device 102 within a threshold amount of time such as 1 day, 1 hour, 10 minutes, or other suitable threshold amount of time), that an instrument device 102 should be depowered, that one or more users of an instrument device 102 should intervene to check on one or more samples associated with instrument device 102, or any suitable action.

Monitoring and analytics system 110 may determine, in accordance with an exemplary second rule, that two out of three data points exceeding a two standard deviation threshold from the target value in the same direction (e.g., two out of three values exceed +/−two standard deviations from the target value) may constitute a medium shift. In response to a medium shift, monitoring and analytics system 110 may transmit a message to a technician with medium urgency, may add an entry to one or more verbose log files (e.g., indicating the occurrence of the medium shift, the time and/or data points at which it occurred, or any users associated with the instrument device 102 at the time of the medium shift), or may add an indication to a suitable health dashboard.

Monitoring and analytics system 110 may determine, in accordance with an exemplary third rule, that a predetermined number of consecutive data points increasing or decreasing in the same direction (e.g., each data point is further from the target value than the previous data point) constitutes a trend, which may be indicative of a developing problem or condition. For example, a trend may be defined as six consecutive data points increasing or decreasing in the same direction.

In response to determining that a trend has occurred in the time series data, monitoring and analytics system 110 may display a graph of the data on a health dashboard for instrument device 102 and may emphasize the graph such that the trend is more easily apparent to a person looking at the graph. For example, monitoring and analytics system 110 may highlight the times during which the data points have fit the definition of a trend. Monitoring and analytics system 110 may add an entry to an instrument-specific log for instrument device 102 indicating that a trend occurred in the data (including, for example, times during which the trend occurred, the values for the data points during which the trend occurred, one or more users associated with instrument device 102 and/or operation of instrument device 102 when the trend occurred, etc.).

FIG. 21 illustrates time series temperature data ingested by monitoring and analytics system 110 from temperature sensor 126 disposed in a biological sample freezer and presented as a dashboard 2100 on a display, e.g., on one or more of information devices 1810a-c. Monitoring and analytics system 110 may determine a target value of −83.6° C., a one standard deviation threshold value of 0.6° C., a two standard deviation threshold value of 1.2° C., and a three standard deviation threshold value of 1.8° C.

FIG. 21 shows a plot of time series temperature data starting on Monday May 15, 2023, and continuing through Friday Jun. 2, 2023. Dashboard 2100 includes instances of monitoring and analytics system 110 identifying that one or more rules have been met.

At times indicated by 2110, monitoring and analytics system 110 identifies that a medium shift has occurred in response to determining that two out of three temperature data points have exceeded a +/−two standard deviation threshold. Accordingly, monitoring and analytics system 110 may take any suitable action in response to identifying a medium shift has occurred, such as transmitting a message to one or more users associated with instrument device 102 at the time of the medium shift. Monitoring and analytics system 110 may query structured utilization data 109, raw utilization data 111, a log file, or any suitable source of information to determine the one or more users associated with instrument device 102 at the time of the medium shift.

At times indicated by 2120, monitoring and analytics system 110 identifies that a large shift has occurred due to a temperature data point exceeding +/−three standard deviations from the target value of −83.6° C. Accordingly, monitoring and analytics system 110 may take any suitable action in response to identifying a large shift has occurred, such as transmitting a message to one or more users associated with instrument device 102 at the time of the large shift and transmitting a message in the form of an urgent service ticket to a technician. Monitoring and analytics system 110 may query structured utilization data 109, raw utilization data 111, a log file, or any suitable source of information to determine the one or more users associated with instrument device 102 and/or one or more technicians responsible for instrument device 102 at the time of the large shift.

At times indicated by 2130, monitoring and analytics system 110 identifies that a trend has occurred due to at least six data points of the time series temperature data increasing in a row. Accordingly, monitoring and analytics system 110 may take any suitable action in response to identifying a trend has occurred, such as backing up data on one or more of instrument device 102 or an associated support device 104, transmitting a message to one or more users associated with instrument device 102 at the time of the trend, and/or transmitting a message in the form of a service ticket to a technician. Monitoring and analytics system 110 may query structured utilization data 109, raw utilization data 111, a log file, or any suitable source of information to determine the one or more users associated with instrument device 102 and/or one or more technicians responsible for instrument device 102 at the time of the trend.

Monitoring and analytics system 110 may additionally or alternatively identify a manually determined or preprogrammed value, such as a significant value related to one or more samples contained within the freezer. At the time indicated by 2140, a temperature monitored by temperature sensor 126 has exceeded a predetermined value of −80° C., which triggers a response by monitoring and analytics system 110. Any suitable response may be taken by monitoring and analytics system 110 to a sensor value exceeding a manually determined value, including backing up data on one or more of instrument device 102 or an associated support device 104, transmitting a message to one or more users associated with instrument device 102 when the predetermined value was exceeded, transmitting a message in the form of a service ticket to a technician, generating an alert (e.g., a visual alert displayed on a suitable display such as information devices 1810a-c, an audio alert such as a siren or similar tone emitted in a lab or office space associated with instrument device 102, a notification sent to a device such as a text message, an email, a haptic alert, or the like), or any suitable response.

A dashboard 2100 may further include one or more icons indicating a severity of an event. For example, severity icons 2150, 2152, and 2154 show differing severity levels in response to events 2110, 2120, and 2140 respectively. For example, severity icon 2150 may indicate a medium shift, severity icon 2152 may indicate a large shift, and severity icon 2154 may indicate a manually programmed threshold has been exceeded.

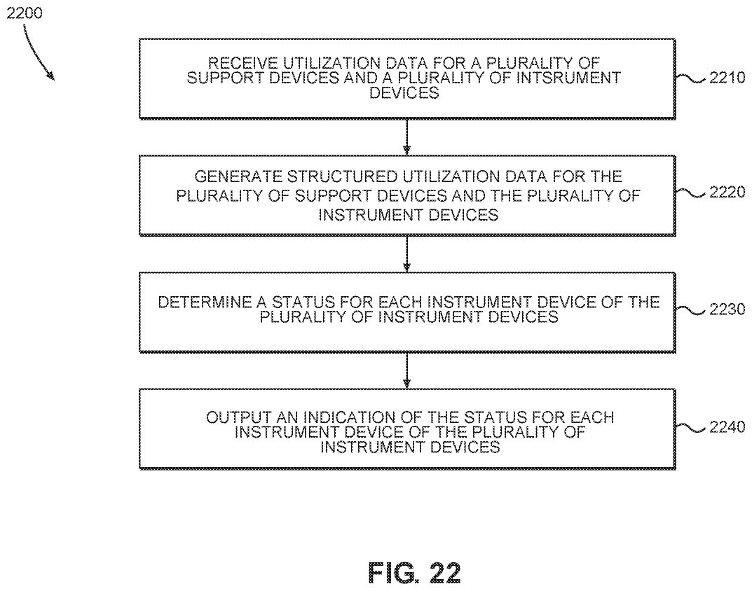

FIG. 22 shows a flowchart of an example method 2200 for improved instrument monitoring and analytics. The method 2200 may be performed in whole or in part by a single computing device, a plurality of computing devices, and the like. For example, the steps of the method 2200 may be performed by a computing device within or associated with the monitoring and analytics system 110 shown in FIG. 1 and/or a computing device in communication with any of the devices/entities of the system 100.

At step 2210, a computing device may receive utilization data (e.g., raw utilization data 111) for each support device of a plurality of support devices and a plurality of instrument devices. Each support device of the plurality of support devices may be in communication with (e.g., communicably coupled to) at least one respective instrument device of the plurality of instrument devices. The plurality of support devices may receive input data from the plurality of instrument devices (e.g., the “input data” described herein).

The utilization data and/or the structured utilization data for each support device of the plurality of support devices may be based on the input data received from the plurality of instrument devices. For example, the utilization data and/or the structured utilization data may be associated with execution of at least one instrument application executing on each instrument device of the plurality of instrument devices. That is, the utilization data and/or the structured utilization data for each support device may be associated with that support device's control/operation of the corresponding instrument device as it collects and/or generates the corresponding input data. For example, the utilization data and/or the structured utilization data for each support device may be associated with that support device receiving, processing, and/or sending the input data (e.g., for storage of the input data at the monitoring and analytics system 110). The input data associated with a particular instrument device may be received by the corresponding support device via the at least one instrument application executing on the particular instrument device. For example, the support device may execute at least one support device application (e.g., support device application 107) that facilitates the support device operating/controlling the instrument device and receiving the corresponding input data.

At step 2220, the computing device may generate structured utilization data for each support device of the plurality of support devices and each instrument device of the plurality of instrument devices. The computing device may generate the structured utilization data for each support device of the plurality of support devices and each instrument device of the plurality of instrument devices based on the utilization data for each support device of the plurality of support devices and a modified database schema. For example, the structured utilization data may comprise the plurality of fields. The structured utilization data may be generated, in some examples, by a support device(s). For example, a first support device of the plurality of support devices may generate the structured utilization data associated with the first support device based on the utilization data associated with the first support device and/or the modified database schema. Additionally, or in the alternative, the database associated with the plurality of support devices may generate the structured utilization data based on the utilization data associated with each support device of the plurality of support devices and/or the modified database schema.

At step 2230, the computing device may determine a status for each instrument device of the plurality of instrument devices, The determination may be based on at least one query associated with the structured utilization data, and the at least one query may be associated with at least one field of the plurality of fields. For example, the status may include an idle state of a respective support device for each instrument device, a connection status of the respective support device for each instrument device, an operational status for each instrument device of the plurality of instrument devices, or a status of an experiment associated with each instrument device of the plurality of instrument devices. In an embodiment, the status may be determined based on a processor usage of each support device associated with the respective instrument device and/or a memory usage of each support device associated with the respective instrument device.

At step 2240, the computing device may output an indication of the status for each instrument device of the plurality of instrument devices via an information device. An information device may include a display, screen, touchscreen, graphical user interface, and/or speaker. In some example embodiments, the information device may be dimensioned, oriented and/or positioned in a given environment, based at least in part on the physical floorplan of the environment, to effectively convey information (e.g., the indication of the status of respective instrument devices) over a significant distance and/or over a substantial portion of the environment (e.g., so as to facilitate communication of the information to multiple recipients occupying the environment).

For example, the computing device may output the health indicator for each instrument device of the plurality of instrument devices via the information device. The computing device may identify a location of each instrument device of the plurality of instrument devices. Outputting an indication of the status for each instrument device of the plurality of instrument devices may further include displaying an indication of the status for each instrument device with a representation of the location of each instrument device.

The at least one field may be associated with, or indicative of, processor usage of each support device of the plurality of support devices. Determining the utilization rate for each instrument device of the plurality of instrument devices may comprise determining that the processor usage, indicated by or associated with at least one field, meets or exceeds a configurable threshold value. The at least one field within the structured utilization data for each support device may be indicative of the processor usage for the corresponding support device. The configurable threshold value may be based on a processor usage associated with an idle state of the support device. Other examples are possible as well.

Additionally, or in the alternative, the at least one field may be associated with, or indicative of, memory usage of each support device of the plurality of support devices. Determining the utilization rate for each instrument device of the plurality of instrument devices may comprise determining that the memory usage, indicated by or associated with at least one field, meets or exceeds a configurable threshold value. The at least one field within the structured utilization data for each support device may be indicative of the memory usage for the corresponding support device. The configurable threshold value may be based on a memory usage associated with an idle state of the support device. At step 2250, the computing device may output an indication of the utilization rate for each instrument device of the plurality of instrument devices. For example, the computing device may output an indication of the utilization rate for each instrument via a graphical user interface, such as one or more of the dashboards described herein.

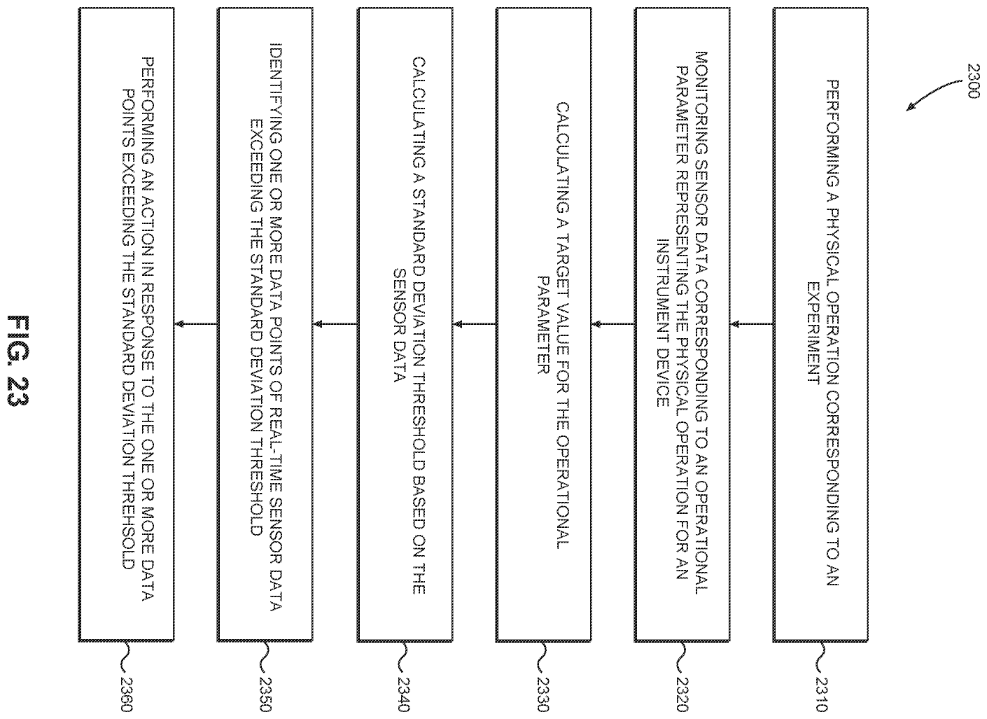

FIG. 23 shows a flowchart of an example method 2300 for monitoring an instrument device. The method 2300 may be performed in whole or in part by a single computing device, a plurality of computing devices, and the like. For example, the steps of the method 2300 may be performed by a computing device within or associated with the monitoring and analytics system 110 shown in FIG. 1 and/or a computing device in communication with any of the devices/entities of the system 100.

At step 2310, an operation is performed by an instrument device. The operation may include increasing or decreasing a temperature of an object, moving, spinning, lifting, shaking, rotating, tilting, stretching, or compressing an object, applying a voltage to an object, applying a current to an object, emitting electromagnetic radiation at an object, or define a frequency of an object. An experiment may be a scientific experiment, an in vivo animal experiment, a biological experiment, an in vitro experiment, or any suitable experiment.

At step 2320, a support device monitors sensor data corresponding to an operational parameter representing the operation for the instrument device. The operational parameter may relate to the operation performed by the instrument device. The operational parameter may include a temperature, voltage, current, frequency, pH, humidity, moisture content, fluid level, velocity, mass, weight, dimension, position, orientation, angle, rotation, acceleration, or the like.

At step 2330, the support device calculates a target value for the operational parameter based on the sensor data. The support device may calculate the target value as an average, such as an average using historical data points or a moving average using the most recent X data points, where X is any suitable value such as 100, 1000, 10,000, 100,000, any value in between the foregoing values, or any value greater or less than the foregoing values. In an embodiment, a target value may be calculated based on a manual entry of a value by a person or other entity.

At step 2340, the support device calculates a standard deviation threshold based on the sensor data. The standard deviation threshold may include one standard deviation, two standard deviations, three standard deviations, or any suitable number of standard deviations. The support device may calculate the standard deviation threshold using historical data points or a moving average using the most recent X data points, where X is any suitable value such as 100, 1000, 10,000, 100,000, any value in between the foregoing values, or any value greater or less than the foregoing values. A number of data points used to calculate a standard deviation threshold may be the same as the number of data points used to calculate the target value, or may be different. In an embodiment, the support device may calculate the standard deviation using all available historical data points.

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for managing a plurality of instrument devices communicatively coupled to respective support devices of a plurality of support devices, the method comprising:
receiving, from a plurality of support devices and a plurality of instrument devices, utilization data for each support device of the plurality of support devices and each instrument device of the plurality of instrument devices, the plurality of support devices being communicably coupled to the plurality of instrument devices, wherein the utilization data is associated with execution of at least one instrument application on each instrument device of the plurality of instrument devices, and wherein each support device controls the instrument device to which that support device is communicably coupled via the at least one instrument application executing on that instrument device;
generating structured utilization data for each support device of the plurality of support devices and each instrument device of the plurality of instrument devices, wherein the structured utilization data comprises a plurality of fields;
determining, based on at least one query associated with the structured utilization data, a status for each instrument device of the plurality of instrument devices, wherein the at least one query is associated with at least one field of the plurality of fields; and
outputting, via an information device, an indication of the status for each instrument device of the plurality of instrument devices.

1. The method of claim 1, wherein the status comprises an idle state of a respective support device for each instrument device, a connection status of the respective support device for each instrument device, an operational status for each instrument device of the plurality of instrument devices, or a status of an operation associated with each instrument device of the plurality of instrument devices.

2. The method of claim 1, further comprising at least one of:
generating, by a first support device of the plurality of support devices, the structured utilization data associated with the first support device based on the utilization data associated with the first support device; or
generating, by a database associated with the plurality of support devices, the structured utilization data based on the utilization data associated with each support device of a plurality of support devices.

3. The method of claim 1, wherein the at least one field of the plurality of fields is associated with a processor usage for each support device of the plurality of support devices, and wherein the structured utilization data for each support device is indicative of the processor usage for that support device.

4. The method of claim 4, wherein determining the status for each instrument device of the plurality of instrument devices comprises:
determining, based on a configurable threshold value associated with the at least one field, that the processor usage indicated by the structured utilization data meets or exceeds the configurable threshold value; and
determining, based on the processor usage meeting or exceeding the configurable threshold value, the status of the instrument device corresponding to each support device of the plurality of support devices.

5. The method of claim 5, wherein the configurable threshold value is based on at least one of a processor usage associated with an idle state of the support device, a connection status of the support device, or data contained in an instrument-specific log file.

6. The method of claim 1, wherein the at least one field of the plurality of fields is associated with memory usage of each support device of the plurality of support devices, and wherein the structured utilization data for each support device is indicative of the memory usage for the that support device.

7. The method of claim 7, wherein determining the status for each instrument device of the plurality of instrument devices comprises:
determining, based on a configurable threshold value associated with the at least one field, that the memory usage indicated by the structured utilization data meets or exceeds the configurable threshold value; and
determining, based on the memory usage meeting or exceeding the configurable threshold value, the status of the instrument device corresponding to each support device of the plurality of support devices.

8. The method of claim 8, wherein the configurable threshold value is based on a memory usage associated with an idle state of the support device.

9. The method of claim 1, further comprising:
identifying a location of each instrument device of the plurality of instrument devices; and
wherein outputting, via an information device, an indication of the status for each instrument device of the plurality of instrument devices comprises displaying an indication of the status for each instrument device with a representation of the location of each instrument device.

10. A method for managing an instrument device, the method comprising:
performing, by an instrument device, a operation;
monitoring, by a support device communicatively coupled to the instrument device, sensor data corresponding to an operational parameter representing the operation for the instrument device;
calculating, by the support device, a target value for the operational parameter based on the sensor data;
calculating, by the support device, a standard deviation threshold based on the sensor data; and
identifying, by the support device, one or more data points of real-time sensor data exceeding the standard deviation threshold.

11. The method of claim 11, wherein performing, by the support device, an action in response to the one or more data points of real-time sensor data exceeding the standard deviation threshold comprises adjusting, by the support device, the operation in response to the one or more data points of real-time sensor data exceeding the standard deviation threshold.

12. The method of claim 12, wherein adjusting, by the support device, the operation in response to the one or more data points of real-time sensor data exceeding the standard deviation threshold comprises terminating the operation.

13. The method of claim 13, wherein the standard deviation threshold comprises two standard deviations.

14. The method of claim 12, wherein adjusting, by the support device, the operation in response to the one or more data points of real-time sensor data exceeding the standard deviation threshold comprises controlling the operation of the instrument device to produce a reversal of the real-time sensor data.

15. The method of claim 11, wherein performing, by the support device, an action in response to the one or more data points of real-time sensor data exceeding the standard deviation threshold comprises transmitting, by the support device, a notification to one or more persons associated with the operation, the notification indicating that the one or more data points of real-time sensor data has exceeded the standard deviation threshold.

16. The method of claim 16, wherein the standard deviation threshold comprises one standard deviation.

17. The method of claim 16, wherein the notification comprises a visual indication that the one or more data points of real-time sensor data has exceeded the standard deviation threshold.

18. The method of claim 16, wherein the notification comprises an auditory indication that the one or more data points of real-time sensor data has exceeded the standard deviation threshold.

19. The method of claim 16, wherein transmitting, by the support device, a notification to one or more persons associated with the operation comprises outputting, via an information device, an indication of a status for the instrument device with a representation of a location of the instrument device.
